(19) United States
(12) Patent Application Publication
(10) Pub. No.: US 2020/0353419 A1
(43) Pub. Date: Nov. 12, 2020

(54) MEMBRANE EMULSIFICATION DEVICE FOR MICROSPHERE CREATION

(71) Applicant: Dauntless 1, Inc., San Diego, CA (US)

(72) Inventors: Olivier LAURENT, San Diego, CA (US); Joel F. MARTIN, Del Mar, CA (US); Bradley J. SARGENT, Mission Viejo, CA (US); Andrew SCHERER, Trabuco Canyon, CA (US); Brian MCMANUS, Somerville, MA (US)

(73) Assignee: Dauntless 1, Inc., San Diego, CA (US)

(21) Appl. No.: 16/766,032

(22) PCT Filed: Nov. 21, 2018
(86) PCT No.: PCT/US2018/062311

§ 371 Date: May 21, 2020

Related U.S. Application Data
(60) Provisional application No. 62/590,155, filed on Nov. 22, 2017.
(60) Provisional application No. 62/653,414, filed on Apr. 5, 2018.
(60) Provisional application No. 62/689,738, filed on Jun. 25, 2018.

(51) Int. Cl. B01D 63/08; B01D 71/02; B01D 71/04; B01D 69/02; B01F 3/08; B01F 5/04; A61K 9/50; A61K 38/31

(52) U.S. Cl. CPC B01D 63/088; B01D 63/087; B01D 71/022; B01D 71/04; B01D 69/02; B01D 2315/10; B01F 5/0478; A61K 9/5031; A61K 38/31; B01D 2325/028; B01F 3/0811

(57) ABSTRACT

The present disclosure is directed to cross-flow membrane emulsification devices. The devices disclosed herein can have a continuous phase plate, a dispersed phase plate, an outlet, and a chamber. The chamber is located between the continuous phase plate and the dispersed phase plate and is bisected by a membrane with a plurality of pores. The chamber can include at least one channel on a first side of the membrane formed from at least one groove in the continuous phase plate and the membrane. In addition, the chamber can also include a cavity on a second side of the membrane formed in the dispersed phase plate.

### CROSS-REFERENCE TO RELATED APPLICATIONS

This application claims the benefit of U.S. Provisional Application No. 62/590,155, filed on Nov. 22, 2017, U.S. Provisional Application No. 62/653,414, filed on Apr. 5, 2018, and U.S. Provisional Application No. 62/689,738, filed on Jun. 25, 2018, which are herein incorporated by reference and for all purposes.

### FIELD OF THE DISCLOSURE

This disclosure relates to membrane emulsification devices. More specifically, this disclosure relates to cross-flow membrane emulsification devices for the creation of microspheres without any moving device components.

### BACKGROUND

Membrane emulsification refers to a technique for creating drops of one liquid (dispersed phase) in another (continuous phase). Specifically, a dispersed phase can be forced through pores in a membrane directly into the continuous phase. Droplets of the dispersed phase can be formed and detached at the end of the pores with a drop-by-drop mechanism.

On a side of the membrane opposite the dispersed phase, the continuous phase can be applying a shear stress to the membrane. The shear stress can help detach the dispersed droplets from the membrane so that they do not coalesce on the membrane surface. This shear force can be created by a stirrer on the continuous phase side of the membrane or various oscillation/pulsation mechanisms. However, such membrane emulsification systems require movable or electrical components. In addition, many membrane emulsification systems are batch processes, wherein the concentration of both the dispersed phase and continuous phase changes over time making it difficult for scaling to manufacturing processes.

### SUMMARY

Provided are cross-flow membrane emulsification devices for the creation of microspheres without any moving or electrical device parts (besides the external pumps feeding liquid to the device). The devices disclosed herein can provide a plurality of microspheres with a narrow and more uniform size distribution. Furthermore, the devices disclosed herein are continuous flow devices that are easily scalable to a large-scale manufacturing process. The devices disclosed herein can be easily scalable due to the geometry and design of the devices. For example, the devices herein are more scalable because having more channels in the device do not change the flow parameters.

In addition, the devices disclosed herein can be easily cleaned and/or sterilized in place or out of place. The devices disclosed herein can be used for applications that require the device to be used in a GMP validated aseptic manufacturing setting. As such, the devices can have no void spaces, dead ends/loops, or inaccessible spaces that would be difficult to clean, sanitize, or sterilize.

In some embodiments, a device includes a continuous phase plate comprising a continuous phase inlet; a dispersed phase plate comprising a dispersed phase inlet; an outlet; and a chamber located between the continuous phase plate and the dispersed phase plate that is bisected by a membrane comprising a plurality of pores, wherein the chamber includes at least one channel on a first side of the membrane formed from at least one groove in the continuous phase plate and the membrane, wherein the at least one channel is fluidly connected between the continuous phase inlet and the outlet; and a cavity on a second side of the membrane formed in the dispersed phase plate that is fluidly connected between the dispersed phase inlet and the plurality of pores in the membrane.

In some embodiments, the at least one channel extends in a direction transverse (i.e., perpendicular) to a flow of a dispersed phase through the plurality of pores. In some embodiments, the continuous phase plate comprises the outlet. In some embodiments, the dispersed phase plate comprises a dispersed phase outlet. In some embodiments, the continuous phase plate comprises at least two grooves. In some embodiments, the chamber comprises at least two channels on the first side of the membrane formed from the at least two grooves in the continuous phase plate and the membrane. In some embodiments, the membrane is removably attached to the dispersed phase plate and/or the continuous phase plate is removably attached to the dispersed phase plate. In some embodiments, the dispersed phase plate comprises a notch and the membrane is mounted in the notch. In some embodiments, the dispersed phase plate comprises stainless steel. In some embodiments, the continuous phase plate comprises stainless steel. In some embodiments, the membrane comprises alignment holes for mounting on the dispersed phase plate. In some embodiments, the membrane comprises stainless steel, tantalum, tungsten, molybdenum, manganese, tin, zinc, or an alloy thereof. In some embodiments, the membrane comprises porous glass or a ceramic. In some embodiments, one or more pores of the plurality of pores has a size between 10-50 microns. In some embodiments, one or more pores of the plurality of pores has a size between 10-20 microns. In some embodiments, the plurality of pores are uniformly sized. In some embodiments, the continuous phase plate, the dispersed phase plate, and the membrane are immobile. In some embodiments, the device does not have any moving device components. In some embodiments, a pressure drop between the continuous phase inlet and the outlet is smaller than the average pressure difference between the dispersed phase side of the membrane and the continuous phase side of the membrane. In some embodiments, a height or width of the at least one channel increases in a direction from the continuous phase inlet to the outlet. In some embodiments, flow in the dispersed phase channel induces a pressure drop about equal to the pressure drop in the continuous phase channel.

In some embodiments, a method of forming microspheres includes flowing a continuous phase through at least one channel of a chamber located between a continuous phase plate and a dispersed phase plate, the chamber bisected by a membrane comprising a plurality of pores, wherein the at least one channel is on a first side of the membrane and is formed from at least one groove in the continuous phase plate and the membrane; forcing, on a second side of the membrane, a dispersed phase through the plurality of pores such that the dispersed phase enters into the continuous phase in a direction that is perpendicular to the continuous phase flow in the at least one channel, wherein forcing the dispersed phase through the plurality of pores into the continuous phase forms a plurality of microspheres comprising the dispersed phase.

In some embodiments, a median diameter of the plurality of microspheres is between 5-100 microns. In some embodiments, a median diameter of the plurality of microspheres is between 10-50 microns. In some embodiments, a median diameter of the plurality of microspheres is between 20-40 microns. In some embodiments, at least about 50%, about 60%, about 70%, about 80%, about 85%, about 90%, about 95%, or about 98% of the plurality of microspheres can have a diameter within 5, 10, 15, or 20 microns above or below the median diameter. In some embodiments, the coefficient of variation of a size distribution of the plurality of microspheres is less than 30%. In some embodiments, the coefficient of variation of a size distribution of the plurality of microspheres is less than 20%. In some embodiments, the coefficient of variation of a size distribution of the plurality of microspheres is between 10-20%. In some embodiments, the perpendicular flow of the continuous phase exerts a shear force at a shear rate at the membrane as the dispersed phase is forced through the plurality of pores. In some embodiments, the shear rate at the membrane is 1,000-25,000 s⁻¹. In some embodiments, the continuous phase comprises an aqueous solvent and the dispersed phase comprises an organic solvent. In some embodiments, the continuous phase further comprises a surfactant. In some embodiments, the dispersed phase further comprises a hydrophobic polymer. In some embodiments, the dispersed phase comprises a therapeutic compound or pharmaceutically acceptable salt thereof. In some embodiments, the dispersed phase comprises a polyol. In some embodiments, a pressure drop between the continuous phase flowing through the at least one channel on the first side of the membrane is smaller than the average pressure difference between the dispersed phase side of the membrane and the continuous phase side of the membrane. In some embodiments, a height or width (or both) of the at least one channel increases in a flow direction of the continuous phase. In some embodiments, flow of the dispersed phase induces a pressure drop about equal to the pressure drop in the continuous phase at least one channel.

In some embodiments, a device includes a continuous phase plate comprising a continuous phase inlet; a dispersed phase plate comprising a plurality of dispersed phase inlets; an outlet; and a chamber located between the continuous phase plate and the dispersed phase plate that is bisected by a membrane comprising a plurality of pores, wherein the chamber comprises: at least one channel on a first side of the membrane formed from at least one groove in the continuous phase plate and the membrane, wherein the at least one channel is fluidly connected between the continuous phase inlet and the outlet; and a cavity on a second side of the membrane formed in the dispersed phase plate that is divided into a plurality of dispersed phase segments, wherein each of the dispersed phase segments are fluidly connected between a dispersed phase inlet and a plurality of pores in a portion of the membrane. In some embodiments, the dispersed phase segments are sequentially arranged along the length of the at least one channel. In some embodiments, a pressure of the dispersed phase in the sequential dispersed phase segments decreases along the length of the at least one channel.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE DRAWINGS

Exemplary embodiments are described with reference to the accompanying figures, in which:

FIG. 1 illustrates an example of a membrane emulsification device disclosed herein.

FIG. 2 illustrates an example of an exploded view of a membrane emulsification device disclosed herein.

FIG. 3A is a cross-section of a membrane emulsification device disclosed herein along axis A in FIG. 1 with the membrane removed.

FIG. 3B is a cross-section of a membrane emulsification device disclosed herein along axis A in FIG. 1 with the membrane in place.

FIG. 3C is a close-up of a channel of FIG. 3B.

FIG. 4 is a cross-section of a membrane emulsification device disclosed herein along axis B in FIG. 1.

FIG. 5 illustrates a cross-section of a membrane emulsification device disclosed herein illustrating a cross-section of a channel along the length of the device.

FIG. 6 illustrates a membrane of a membrane emulsification device disclosed herein.

FIG. 7A illustrates a first example pore configuration of the membrane.

FIG. 7B illustrates a second example pore configuration of the membrane.

FIG. 7C illustrates a third example pore configuration of the membrane.

FIG. 7D illustrates a fourth example pore configuration of the membrane.

FIG. 8A illustrates a size distribution graph for an example of microspheres produced by the device disclosed herein.

FIG. 8B illustrates a size distribution graph for a comparative example of microspheres produced by a Micropore® commercial device.

FIG. 9A illustrates an SEM image of the microspheres created by the device disclosed herein.

FIG. 9B illustrates an SEM image of the microspheres created by the device disclosed herein.

FIG. 9C illustrates an SEM image of the microspheres produced by a Micropore® commercial device.

FIG. 9D illustrates an SEM image of the microspheres produced by a Micropore® commercial device.

FIG. 10 illustrates an example of a membrane emulsification device disclosed herein.

FIG. 11 illustrates an example of a membrane emulsification device disclosed herein.

FIG. 12 illustrates an example of an exploded view of a membrane emulsification device disclosed herein.

FIG. 13 illustrates an example of a dispersed phase plate disclosed herein.

FIG. 14 illustrates an example of a continuous phase plate disclosed herein.

FIG. 15 illustrates an example of a dispersed phase plate side of the a membrane emulsification device disclosed herein.

FIG. 16A is a cross-section of a membrane emulsification device disclosed herein along axis C in FIG. 10.

FIG. 16B is a cross-section of a membrane emulsification device disclosed herein along axis D in FIG. 10.

FIG. 17A is a close-up of the cross-section of FIG. 16A.

FIG. 17B is a close-up of the cross-section of FIG. 16A.

FIG. 18A is an example of a circular membrane.

FIG. 18B is an example of a circular membrane.

FIG. 19 is a simulation using a differential pressure between the dispersed phase and continuous of 0.25 PSI showing the deflection of the circular membrane when that membrane is used for crossflow membrane emulsification.

FIG. 20A is an example of a membrane disclosed herein.

FIG. 20B is an example of a membrane disclosed herein.

FIG. 21 is a simulation using a differential pressure between the dispersed phase and continuous phase of 0.25 PSI showing the deflection of the membrane disclosed herein when that membrane is used for crossflow membrane emulsification.

FIG. 22 is a plot showing the membrane deflection with respect to the pressure for the simulations using a stainless steel circular membrane and the stainless steel membrane disclosed herein.

FIG. 23 is a plot showing the membrane deflection with respect to the pressure for the simulations using a stainless steel membrane disclosed herein and a molybdenum membrane disclosed herein.

FIG. 24 illustrates an example of the flow of the continuous phase and dispersed phase in a membrane emulsification device disclosed herein.

FIG. 25 illustrates an example of the pressure gradient in the continuous phase and the dispersed phase in a membrane emulsification device disclosed herein.

FIG. 26 illustrates an example of an embodiment of a membrane emulsification device disclosed herein with continuous phase inlets and outlets in parallel.

FIG. 27 illustrates an example of an embodiment of a membrane emulsification device disclosed herein with parallel continuous and dispersed phase flow in order to maintain a pressure differential that is constant along the membrane

FIG. 28 is a graph of continuous phase channel width of a membrane emulsification device according to an embodiment disclosed herein.

FIG. 29 illustrates an example of a membrane emulsification device disclosed herein.

FIG. 30 is a cross-section of a membrane emulsification device disclosed herein along axis B in FIG. 29.

FIG. 31 is a cross-section of an embodiment of a membrane emulsification device disclosed herein.

FIG. 32 is a diagram showing the direction flow of the dispersed phase and continuous phase through the membrane emulsification device for example 2.

FIG. 33 shows the different microspheres that are generated at various continuous phase and dispersed phase flow rates for example 2. (A) DP 10 mL/min, CP 3.4 L/min; (B) DP 10 mL/min, CP 3.0 L/min; (C) DP 9 mL/min, CP 2.3 L/min; (D) DP 9 mL/min, CP 1.7 L/min; (E) DP 12 mL/min, CP 1.7 L/min.

FIG. 34 compares the size distribution of microspheres generated with the Micropore Dispersion Cell compared to the microspheres generated using the device disclosed herein for example 2.

FIG. 35 compares the size distribution of microspheres generated with the Micropore Dispersion Cell (1 gram) compared to microspheres generated using the device disclosed herein (both 10 and 30 gram) for example 2.

FIG. 36 shows the microsphere size distribution from two 10 g and one 30 g batches of production for example 3.

FIG. 37 illustrates an example of a membrane emulsification device containing more than 2 channels disclosed herein.

FIG. 38 illustrates the dispersed phase plate side of a membrane emulsification device containing more than 2 channels disclosed herein.

FIG. 39 illustrates a cross section along the length of a membrane emulsification device containing more than 2 channels disclosed herein.

FIG. 40 illustrates a cross section along the width of a membrane emulsification device containing more than 2 channels disclosed herein.

FIG. 41 illustrates an exploded view of a membrane emulsification device containing more than 2 channels disclosed herein.

In the figures, like reference numbers correspond to like components unless otherwise stated. In addition, the Figures are not drawn to scale.

### DETAILED DESCRIPTION

The membrane emulsification devices disclosed herein can create microspheres without any moving or electrical device components. Specifically, the devices disclosed herein are continuous flow devices that are easily scalable to large scale manufacturing processes and can be easily cleaned and/or sterilized.

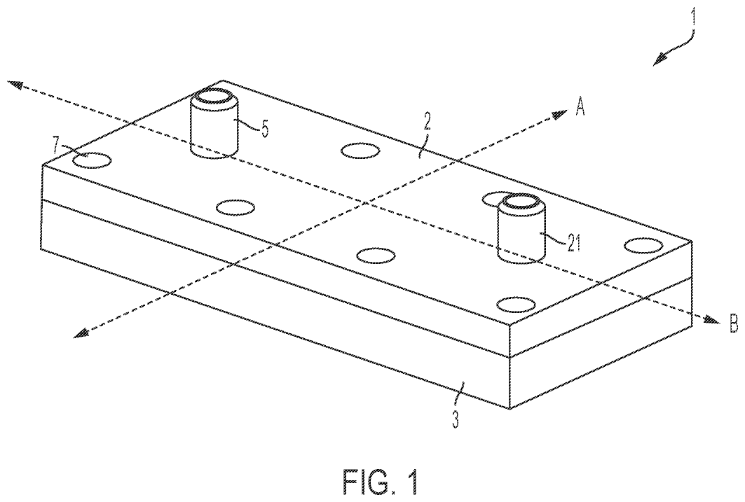

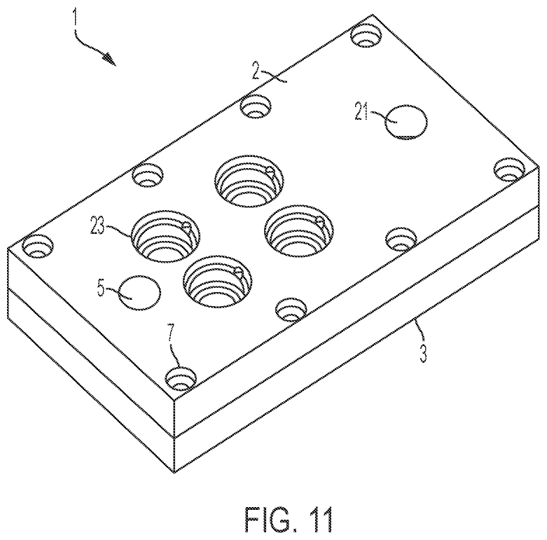

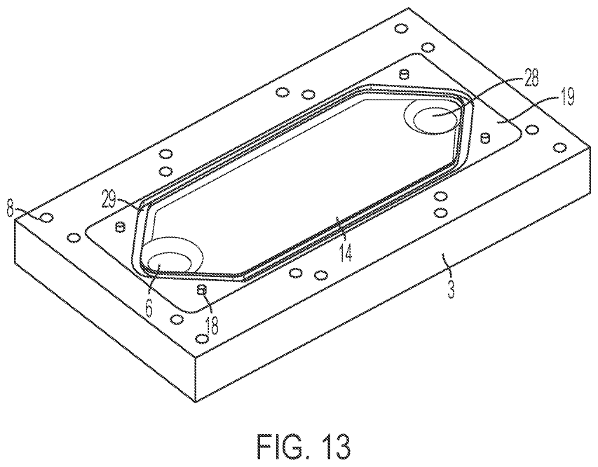

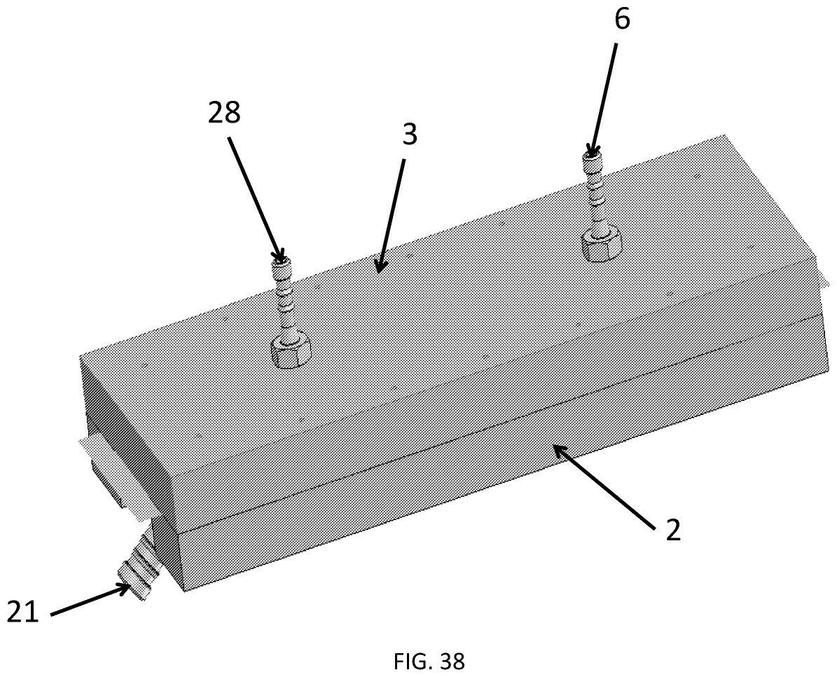

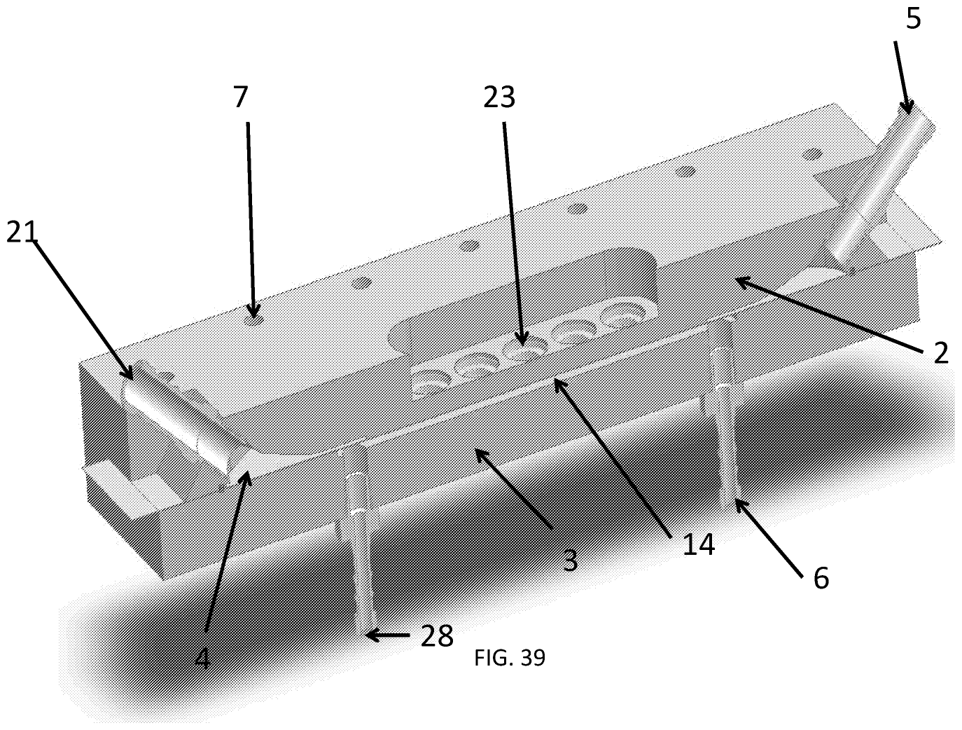

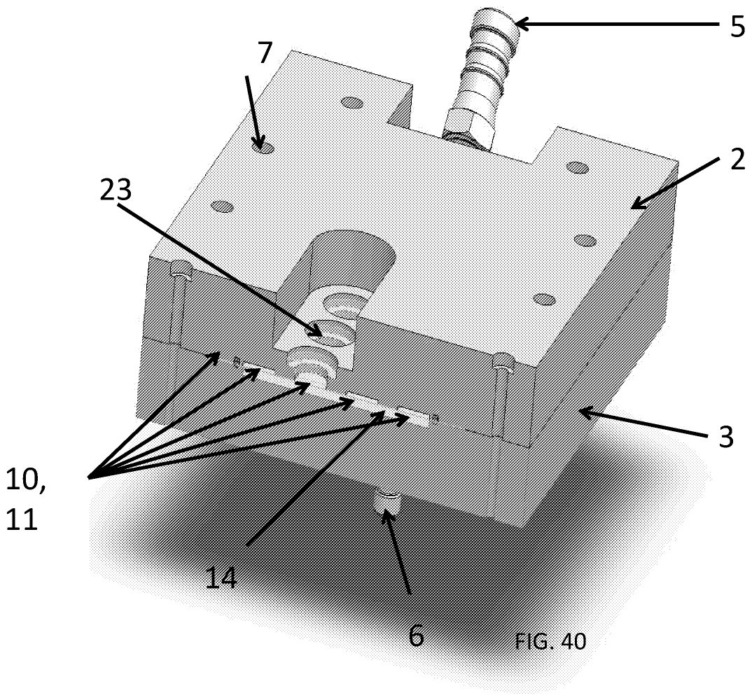

FIGS. 1-5, 10-17B, 24-27, 29-32, and 37-41 illustrate examples of a membrane emulsification device. Membrane emulsification device 1 can include continuous phase plate 2, dispersed phase plate 3, and membrane 4. The continuous phase plate, the dispersed phase plate, and the membrane can be immobile. In other words, the continuous phase plate, the dispersed phase plate, and the membrane may not move during operation of the device. In some embodiments, the device does not include any moving device components. Instead, the only moving things in the device can be the continuous phase and the dispersed phase moving through the device.

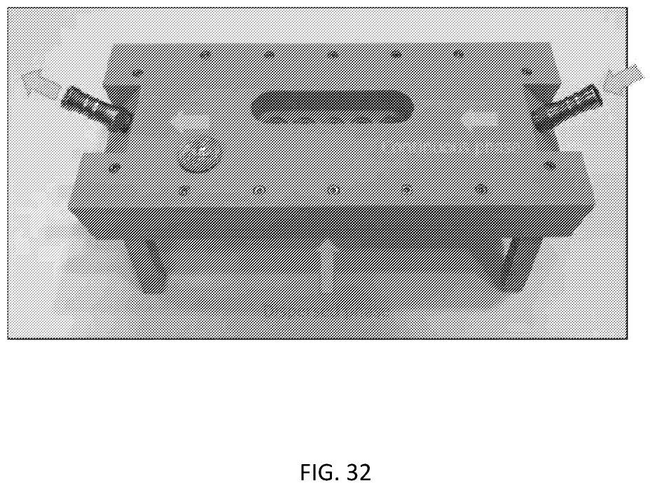

The continuous phase plate can allow a continuous phase to enter into the device. For example, a user of the device can flow a continuous phase through continuous phase inlet 5 such that the continuous phase enters the device through continuous phase plate 2. In some embodiments, the continuous phase inlet is on an outer surface of the continuous phase plate. The continuous phase inlet does not have to be on an outer surface. Instead, the continuous phase inlet can be on any one of the side surfaces of the continuous phase plate. In addition, the continuous phase inlet can be closer to one end of the continuous phase plate than the middle of the plate, as depicted in FIG. 1. In some embodiments, the continuous phase inlet can be on an outer surface that is not on the same plane as the top most outer surface of the continuous phase plate as shown in FIG. 32. As shown in FIG. 32, the continuous phase inlet can be on an outer surface of the continuous phase plate that slants toward the dispersed phase plate. In addition, this slanting surface can slant away from the center of the continuous phase plate. The continuous phase plate can be made out of a material that is solvent resistant for the particular microspheres that are being created. In some embodiments, the continuous phase plate can be made out of stainless steel or polyether ether ketone (“PEEK”).

In some embodiments, the length of the continuous phase plate can be at least about 50 mm, about 75 mm, about 100 mm, about 150 mm, about 300 mm, or about 500 mm. In some embodiments, the length of the continuous phase plate can be about 10-500 mm, about 50-300 mm, or about 75-150 mm. In some embodiments, the width of the continuous phase plate can be at least about 25 mm, about 35 mm, about 50 mm, about 55 mm, about 75 mm, about 100 mm, about 250 mm, about 500 mm, about 1,000 mm, or about 2,000 mm. In some embodiments, the width of the continuous phase plate can be about 10-100 mm, about 15-75 mm, or about 25-75 mm. In some embodiments, the thickness of the continuous phase plate can be about 1-50 mm, about 5-25 mm, or about 10-15 mm.

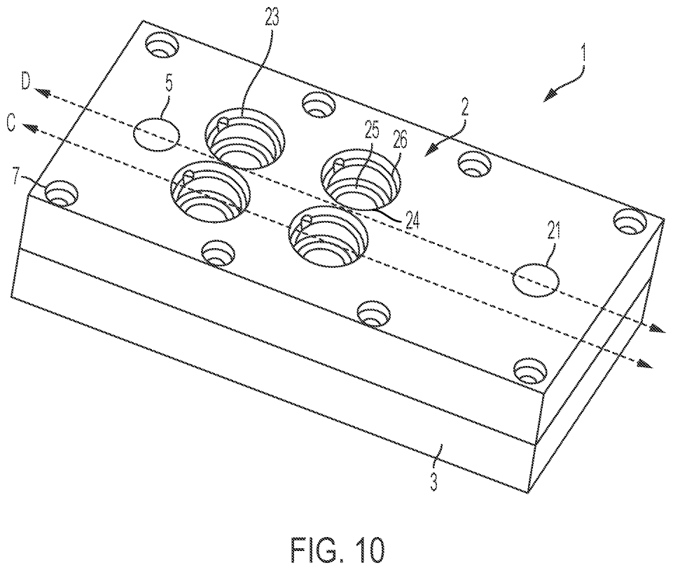

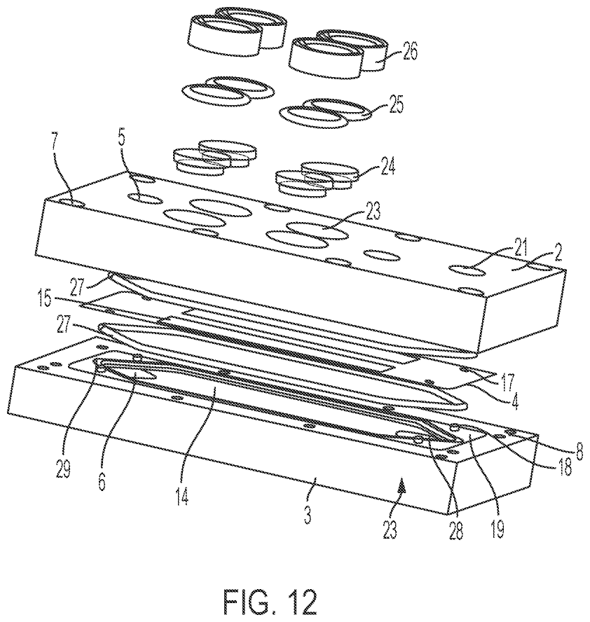

In some embodiments, the continuous phase plate can have at least one viewing window over the membrane so that a user can view the microsphere formation during use. For example, viewing window 23 is shown in FIGS. 10-11 and 41. The at least one viewing window can include transparent windows 24 (e.g., glass), O-rings 25, and/or retainer rings 26 as shown in FIG. 12. The transparent window, O-ring, and retainer ring can all fit inside the viewing window.

The dispersed phase plate can allow a dispersed phase to enter into the device. For example, a user of the device can flow a dispersed phase through dispersed phase inlet 6 such that the dispersed phase enters the device through dispersed phase plate 3. In some embodiments, the dispersed phase inlet is on an outer surface of the dispersed phase plate. The dispersed phase inlet does not have to be on an outer surface. Instead, the dispersed phase inlet can be on any one of the side surfaces of the dispersed phase plate. In addition, the dispersed phase inlet can be centered on the dispersed phase plate, as depicted in FIG. 1. In some embodiments, the dispersed phase inlet can be closer to one end of the dispersed phase plate than the middle of the plate. The dispersed phase plate can be made out of a material that is solvent resistant for the particular microspheres that are being created. In some embodiments, the dispersed phase plate can be made out of stainless steel or PEEK.

In some embodiments, the length of the dispersed phase plate can be at least about 50 mm, about 75 mm, about 100 mm, about 150 mm, about 300 mm, or about 500 mm. In some embodiments, the length of the dispersed phase plate can be about 10-500 mm, about 50-300 mm, or about 75-150 mm. In some embodiments, the width of the dispersed phase plate can be at least about 25 mm, about 35 mm, about 50 mm, about 55 mm, about 75 mm, about 100 mm, about 250 mm, about 500 mm, about 1,000 mm, or about 2,000 mm. In some embodiments, the width of the dispersed phase plate can be about 10-100 mm, about 15-75 mm, or about 25-75 mm. In some embodiments, the thickness of the dispersed phase plate can be about 1-50 mm, about 5-25 mm, or about 10-15 mm. In some embodiments, the combined thickness of the continuous phase plate attached to the dispersed phase plate (i.e., the sandwich of the plates) can be about 1-100 mm, about 5-75 mm, about 10-50 mm, or about 15-30 mm.

FIGS. 1-5 illustrates the membrane emulsification device as being horizontal. However, the membrane emulsification device can be vertical. As such, the force of gravity can supplement the shear force of the downward flow of the continuous phase in removing the microspheres from the membrane. In some embodiments, the continuous phase and/or the dispersed phase can be flowing against gravity.

In some embodiments, the dispersed phase plate can include a bleed valve. The bleed valve can allow the removal of bubbles of air and/or continuous phase that has become trapped on the dispersed phase side of the membrane. Furthermore, the dispersed phase inlet can be connected to a bleed valve. In some embodiments, the bleed valve can be on an outer surface of the dispersed phase plate. The bleed valve can also be on any one of the side surfaces of the dispersed phase plate. For example, the bleed valve can be situated toward the top of the dispersed phase plate (if the device is not horizontal) to improve removal of floating air bubbles or residual low-density fluids.

In some embodiments, the continuous phase plate can be removably attached to the dispersed phase plate. Having the plates be removably attached to one another can allow for quick dismantling of the device for cleaning, maintenance, or any other reason. In addition, removing the continuous phase plate from the dispersed phase plate can allow for access to the membrane for replacement and/or maintenance of the membrane. The continuous phase plate can have alignment holes 7 and the dispersed phase plate can have alignment holes 8. The alignment holes on the continuous phase plate can align up with the alignment holes on the dispersed phase plate. As such, the continuous phase plate can be secured to the dispersed phase plate using these alignment holes. For example, the continuous phase plate can be screwed or nailed to the dispersed phase plate. In some embodiments, the continuous phase plate can be attached to the dispersed phase plate using an adhesive. In some embodiments, the continuous phase plate can be attached to the dispersed phase plate using clamps pressing both plates together.

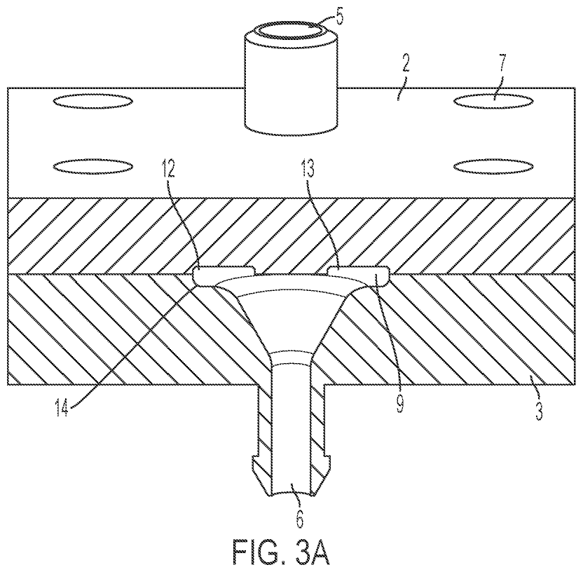

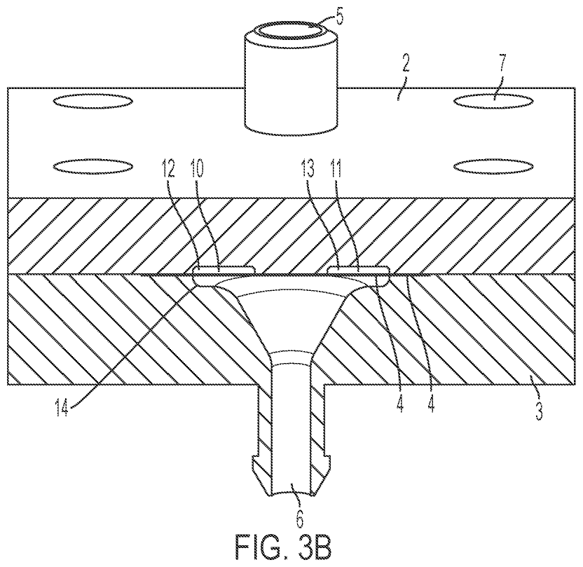

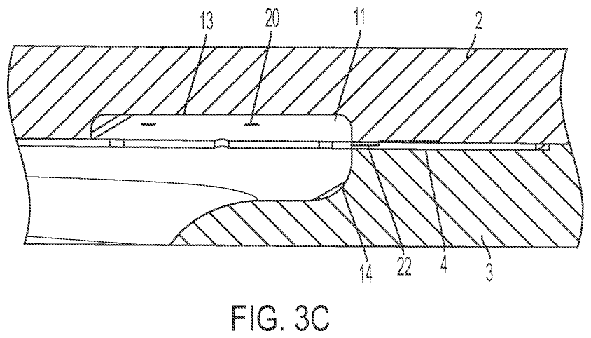

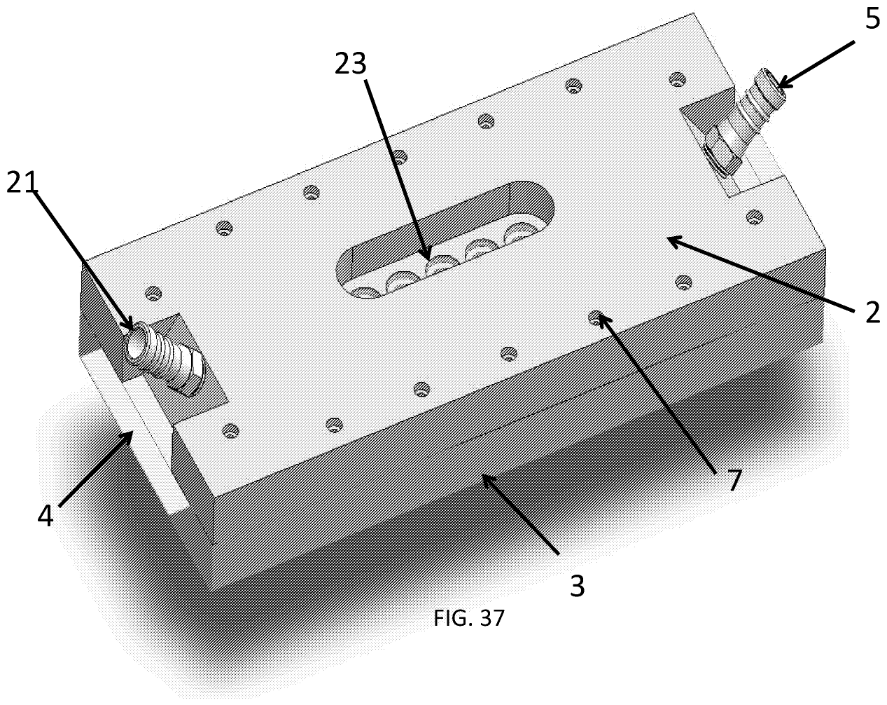

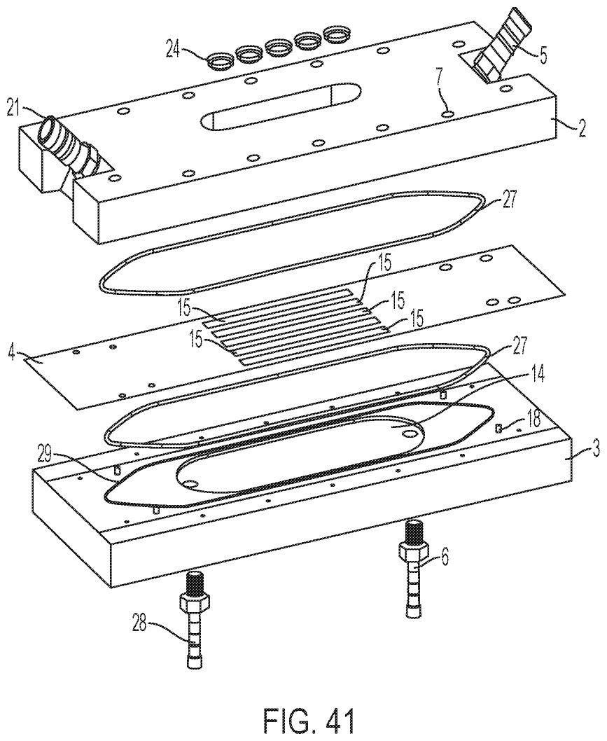

As shown in FIG. 3A which is a cross-section of the device with the membrane removed along axis A in FIG. 1, the inside of the device includes chamber 9 located between continuous phase plate 2 and dispersed phase plate 3. The chamber allows both the dispersed phase and the continuous phase to flow through the device towards the outlet. The chamber can be bisected by membrane 4 as shown in FIG. 3B. The chamber can include at least one channel on a side of the membrane. In some embodiments, the chamber can include at least two channels on a side of the membrane. In some embodiments, the chamber can include a plurality of channels on a side of the membrane. For example, FIG. 3B includes channels 10 and 11. The at least one channel can be formed from at least one groove in the continuous phase plate and the membrane. The at least one groove can be formed in a surface of the continuous phase plate facing the membrane. For example, device 1 includes two grooves 12 and 13 formed in a surface of continuous phase plate 2. As shown in FIG. 3C, groove 13 and membrane 4 form channel 11. In some embodiments, the device can include a plurality of grooves in the continuous phase plate. The plurality of grooves and the membrane can form a plurality of channels in the device. For example, FIGS. 37-41 illustrate a device with five grooves and five channels. The continuous phase can enter through a continuous phase inlet 5 and spread evenly across the five channels 10,11 corresponding to the five regions 15 containing the plurality of pores in membrane 4 illustrated in FIGS. 40-41. In addition, FIG. 40 illustrates that the windows 23 allow vision into only one of the five channels in the device. Furthermore, although FIGS. 3-41 illustrate the continuous phase inlet 5 and the dispersed phase inlet 6 on the same side of the device (and the outlet 21 and dispersed phase outlet 28 on the same side of the device), the inlets and outlets can be switched such that the continuous phase inlet 5 and the dispersed phase outlet 28 are on the same side of the device such that continuous phase and the dispersed phase are flowing counter to one another.

In addition, the continuous phase plate can include at least one ridge 22 on a surface for compression with the membrane. In some embodiments, the continuous plate can include ridges that run the length of the at least one channel on both sides of the at least one channel. The dimensions of the at least one channel can directly impact the microsphere creation as the at least one channel controls the flow of the continuous phase through the device. In addition, as explained below, a pressure gradient can occur in the at least one channel as the continuous phase flows along the length of the membrane. Accordingly, the dimensions of the at least one channel need to be chosen such that the pressure drop across the length of the membrane in the continuous phase is smaller than the average pressure difference between the dispersed phase side of the membrane and the continuous phase side of the membrane.

The at least one channel can have a width of about 1-20 mm, about 2-18 mm, about 5-15 mm, about 8-12 mm, about 9-11 mm, about 5-11 mm, or about 10-11 mm. In some embodiments, the width of the at least one channel is greater than about 20 mm, about 50 mm, about 100 mm, about 500 mm, or about 1000 mm. In some embodiments, the at least one channel can have a width that starts at one of the values disclosed above and increases along the length of the membrane in the continuous phase flow direction. The at least one channel can have a height of about 0.1-20 mm, about 0.1-15 mm, about 0.1-10 mm, about 0.1-5 mm, about 0.1-2 mm, about 0.1-1 mm, about 0.1-0.7 mm, about 0.1-0.5 mm, about 0.1-0.4 mm, or about 0.1-0.3 mm. In some embodiments, the at least one channel can have a height of less than about 20 mm, about 15 mm, about 12 mm, about 10 mm, about 8 mm, about 5 mm, or about 2 mm. In some embodiments, the at least one channel can have a height that starts at one of the values disclosed above and increases along the length of the membrane in the continuous phase flow direction.

Because the at least one groove in the continuous phase plate forms part of the at least one channel, the at least one groove can have a width of about 1-20 mm, about 2-18 mm, about 5-15 mm, about 8-12 mm, about 9-11 mm, about 5-11 mm, or about 10-11 mm. In some embodiments, the width of the at least one groove is greater than about 20 mm, about 50 mm, about 100 mm, about 500 mm, or about 1000 mm. In some embodiments, the at least one groove can have a width that starts at one of the values disclosed above and increases along the length of the membrane in the continuous phase flow direction. In addition, the at least one groove can have a depth into the continuous plate of about 0.1-20 mm, about 0.1-15 mm, about 0.1-10 mm, about 0.1-5 mm, about 0.1-2 mm, about 0.1-1 mm, about 0.1-0.7 mm, about 0.1-0.5 mm, about 0.1-0.4 mm, or about 0.1-0.3 mm. In some embodiments, the at least one groove can have a depth of less than about 20 mm, about 15 mm, about 12 mm, about 10 mm, about 8 mm, about 5 mm, or about 2 mm. In some embodiments, the at least one groove can have a depth that starts at one of the values disclosed above and increases along the length of the membrane in the continuous phase flow direction.

The smaller the space in the at least one channel, the lower volumetric flowrate can be used to maintain a certain shear. As such, a higher concentration of microspheres can be obtained from the device and a higher total throughput. In addition, the smaller the dimensions of the at least one channel, the lower the volumetric flowrate can be of the continuous phase. However, the dimensions of the at least one channel still need to be chosen such that the pressure drop across the length of the membrane in the continuous phase is smaller than the average pressure difference between the dispersed phase side of the membrane and the continuous phase side of the membrane. Furthermore, in some embodiments, the at least one channel can maintain laminar flow of the continuous phase over the membrane.

In some embodiments in which there is more than one channel, the channels can have the same dimensions. In other embodiments in which there is more than one channel, the channels can all have different dimensions or some of the channels can have the same dimensions.

The at least one channel can be fluidly connected between the continuous phase inlet and the outlet. As such, the continuous phase can enter the device through the continuous phase inlet and flow through the at least one channel to the outlet. The at least one channel can extend in the lengthwise direction of the device. In some embodiments, the at least one channel extends perpendicular to at least one of the continuous phase inlet or the dispersed phase inlet. In some embodiments, the distance between the center of the continuous phase inlet to the center of the outlet can be about 10-200 mm, about 25-150 mm, about 50-100 mm, about 55-85 mm, about 60-70 mm, or about 65-70 mm.

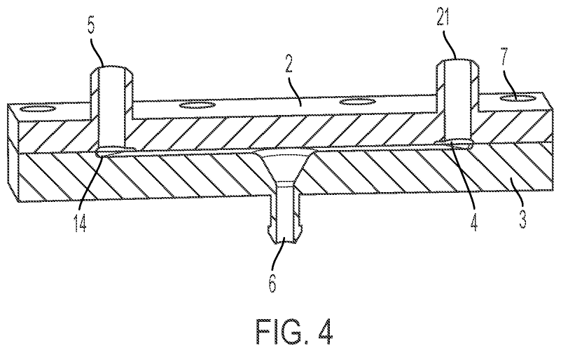

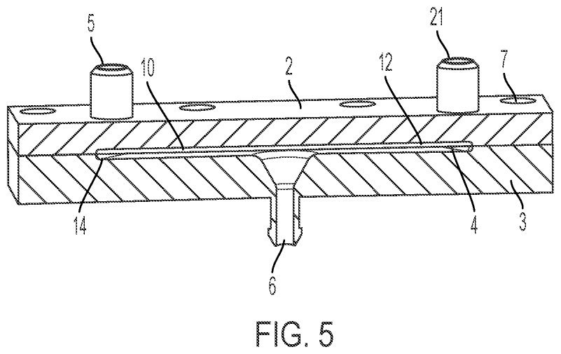

FIG. 4 does not include a channel in the cross-section of the device because channels 10 and 11 of device 1 are not directly on axis B of the device. Instead, they can be on each side of axis B. In contrast to FIG. 4, FIG. 5 illustrates a cross-section of a device illustrating a cross-section of a channel along the length of the device.

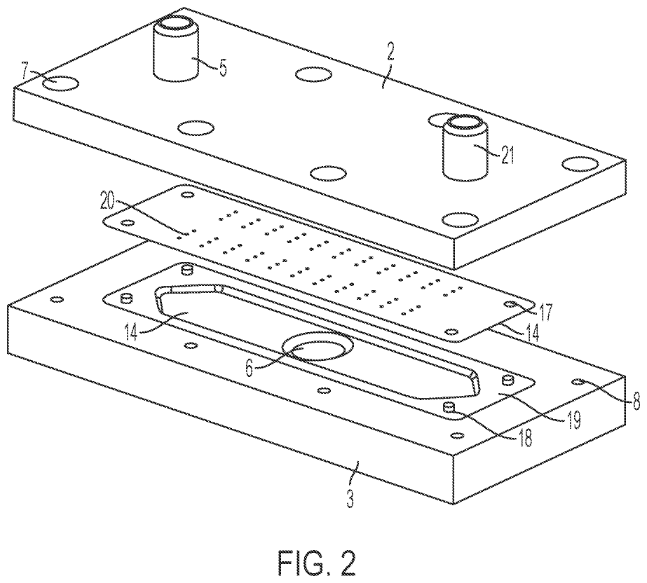

The chamber can also include a cavity on a side of the membrane opposite the at least one channel. For example, FIGS. 2, 4, 5, 12, and 13 illustrate cavity 14. The cavity can be formed in a surface of the dispersed phase plate facing the membrane. In some embodiments, the cavity can have a depth of about 0.1-5 mm, about 0.2-3 mm, about 0.5-2 mm, about 0.75-1.25 mm, or about 1 mm in the dispersed phase plate. In some embodiments, the cavity can have a width of at least about 5 mm, about 10 mm, about 20 mm, about 25 mm, about 30 mm, about 50 mm, about 75 mm, about 100 mm, about 500 mm, or about 1000 mm. In some embodiments, the cavity can have a width of about 1-100 mm, about 5-75 mm, about 10-50 mm, about 15-40 mm, or about 20-30 mm.

The cavity can be fluidly connected between the dispersed phase inlet and the plurality of pores in the membrane. In some embodiments, the cavity can be fluidly connected between the dispersed phase inlet, the plurality of pores in the membrane, and a dispersed phase outlet. As such, a portion of the dispersed phase can enter the device through the dispersed phase inlet and flow through the cavity to the plurality of pores and a portion of the dispersed phase can enter the device through the dispersed phase inlet and flow through the cavity out the dispersed phase outlet. In some embodiments, the distance between the center of the dispersed phase inlet to the center of the dispersed phase outlet can be about 10-200 mm, about 25-150 mm, about 50-100 mm, about 55-85 mm, about 65-75 mm, or about 70-75 mm.

A portion of the dispersed phase can be forced through the pores into the at least one channel where the continuous phase is flowing. The at least one channel can extend in a direction transverse to the flow of the dispersed phase through the plurality of pores such that the dispersed phase enters into the continuous phase in a direction that is perpendicular to the continuous phase flow in the at least one channel.

During membrane emulsification disclosed herein, a dispersed phase can be forced through the pores of a membrane, while the continuous phase flows along the membrane surface in a direction that is perpendicular to the flow of the dispersed phase through the pores. Droplets of the dispersed phase can grow at pore outlets until they reach a certain size and detach. The continuous phase flow can essentially wash these droplets off the membrane and carry them to the outlet. By washing these droplets off of the membrane, the size of these droplets can be controlled.

The size of the droplets (i.e., microspheres) can be determined by a variety of factors. For example, these factors include, but are not limited to, the shear force on the droplet from the flowing continuous phase, differential surface tension between the continuous phase and dispersed phase, density of the two phases, viscoelastic properties of the liquids, the rate of extrusion of the dispersed phase through the membrane, presence of surfactants, and the pore size. The droplets at the pores tend to a form short filaments or cylinders that detach from the membrane surface and then form spheres to minimize surface area after detachment. The filaments pinch off/detach due to the force of the stream and Rayleigh instability. As such, once these droplets detach from the membrane, they can be considered to be microspheres of the dispersed phase. The final size of these microspheres and the size distribution of these microspheres are not only determined by the pore size and size distribution of the pores, but can also be affected by the degree of coalescence, properties of the two phases, and presence of surfactants, both at the membrane surface and in the bulk solution.

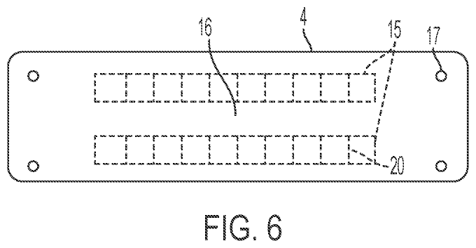

As discussed above, the membrane can include a plurality of pores. In some embodiments, the membrane has the most holes per unit area while still obtaining good uniform particle size distribution. This plurality of pores can be within at least one region of the membrane. For example, FIG. 6 illustrates two regions 15 (dotted lines) with plurality of pores 20. The at least one region of the membrane can correspond to the at least one groove of the continuous phase plate. As such, the at least one groove in the continuous phase plate and the at least one region of the membrane can form the at least one channel in the chamber of the device. Accordingly, the width of the at least one channel can correspond to the width of the at least one region of the membrane. The at least one region can have a width of about 1-20 mm, about 2-18 mm, about 5-15 mm, about 8-12 mm, about 9-11 mm, about 5-10 mm, or about 10 mm. In some embodiments, the width of the at least one region is greater than about 20 mm, about 50 mm, about 100 mm, about 500 mm, or about 1000 mm.

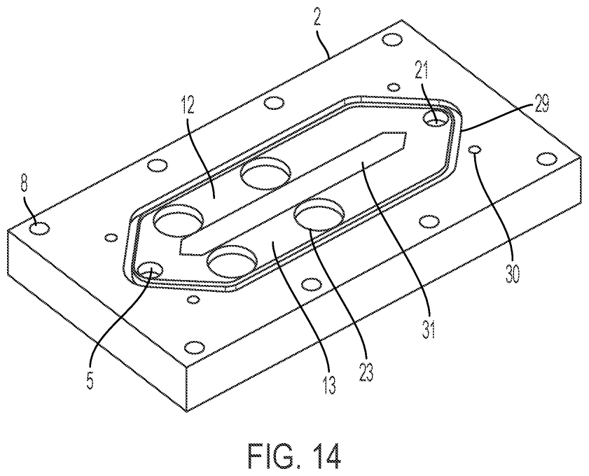

In some embodiments in which there is more than one channel, the regions of the membrane can have the same dimensions. In other embodiments in which there is more than one channel, the regions can all have different dimensions or some of the regions can have the same dimensions. If there is more than one region, the regions can be separated by a gap. FIG. 6 has gap 16 between two regions 15. The gap(s) can have a width of about 1-15 mm, about 2-10 mm, about 4-8 mm, about 5-7 mm, or about 6.5 mm. The gap(s) may not be in contact with the continuous flow through the device as the gap may be outside of the at least one channel. In addition, the gap(s) may not have any pores. The membrane may also have a gap(s) between the at least one region and the edge of the membrane. This gap(s) can be used to clamp down the membrane between the continuous phase plate and the dispersed phase plate. In other words, a portion of the continuous phase plate can be in contact with the gap(s) to help compress the membrane between the dispersed phase plate and the continuous phase plate. These portions of the continuous phase plate that are in contact with the gap(s) can be pillars that divide the channels in the chamber. As illustrated in FIG. 14, the continuous phase entering the continuous phase inlet can be divided by at least one pillar 31 such that the continuous phase will be split between the various grooves/channels. After the continuous phase travels the length of the grooves/channels, the continuous phase in the grooves/channels can then converge at the outlet. Furthermore, these pillars that are in contact with the gap(s) in the membrane can prevent the membrane from deflecting significantly upward from the pressure differential between the dispersed phase (higher pressure) and the continuous phase (lower pressure). In addition, the continuous phase plate and the dispersed phase plate (with the membrane) can be compressed such that the membrane does not move or has minimal movement/deflection when the device is in operation.

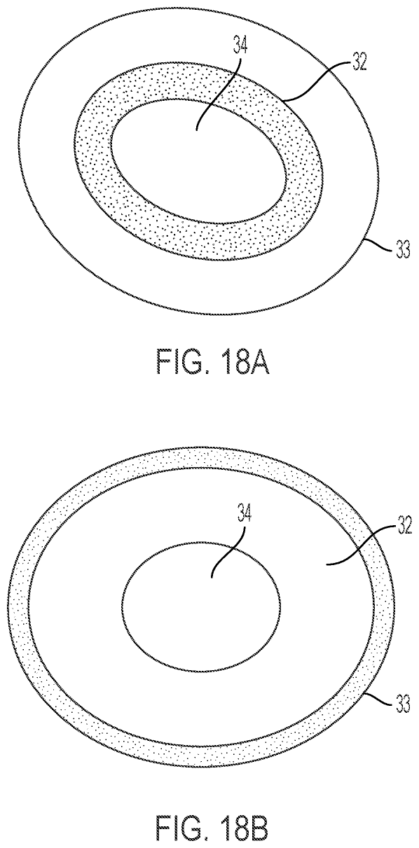

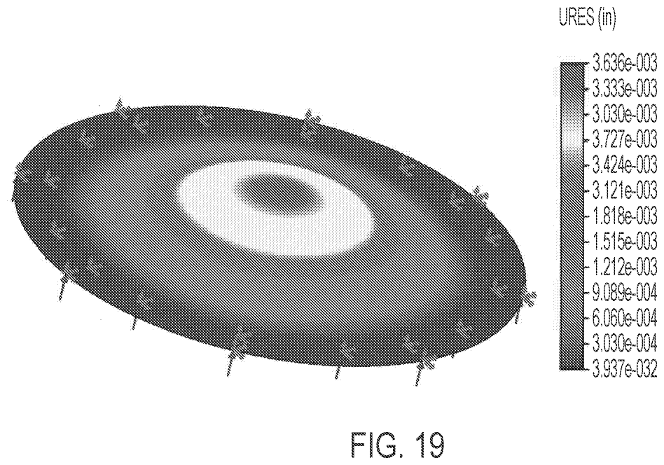

Applicants have discovered that the combination of the pore region(s) and the regions of the membrane that are contacted between the continuous phase plate and the dispersed phase plate can greatly minimize the amount of deflection the membrane experiences compared to other membrane configurations. For example, FIGS. 18A-18B illustrate a circular membrane having interior circular pore region 32 and region contacted/supported 33 between the continuous phase plate and the dispersed phase plate. In addition, region 34 is a region of the membrane without holes that is not supported by between the continuous phase plate and the dispersed phase plate. FIG. 19 is a simulation using a pressure at 0.25 PSI showing the deflection (not to scale) of the membrane of FIGS. 18A-18B when that membrane is used for crossflow membrane emulsification. As shown in FIG. 19, the membrane is significantly deflected upward from the pressure differential between the dispersed phase (higher pressure) and the continuous phase (lower pressure).

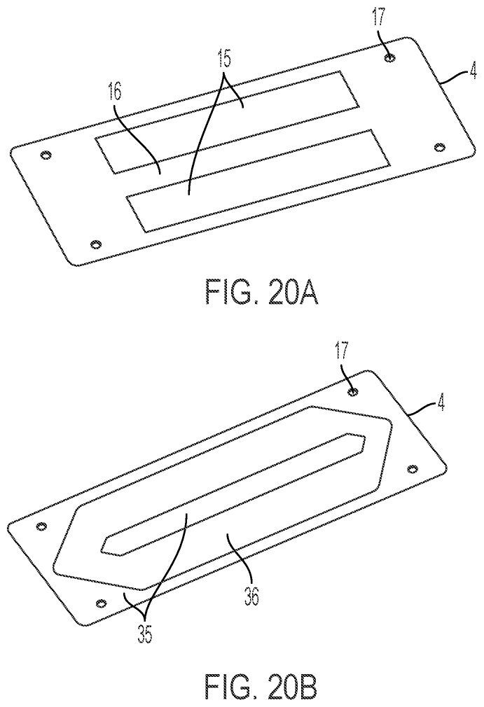

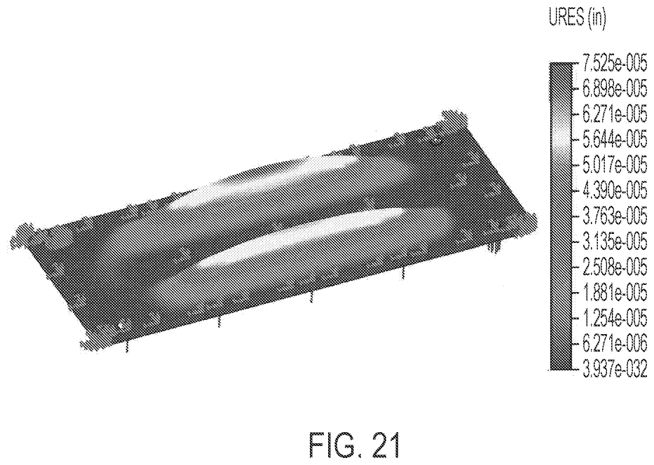

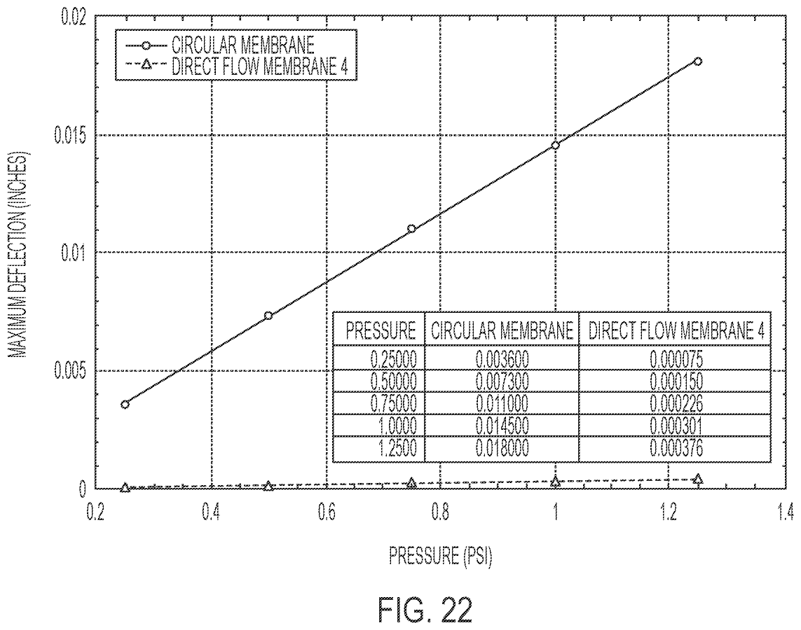

In contrast to the membrane of FIGS. 18A-18B, the device disclosed herein can significantly reduce the deflection on the membrane. FIGS. 20A-20B illustrate membrane 4. Specifically, FIG. 20B illustrates region 35 of membrane 4 that is contacted/supported between the continuous phase plate and the dispersed phase plate and region 36 that is not supported between the continuous phase plate and the dispersed phase plate. Region 36 is the region where the continuous phase flows from the continuous phase inlet through the at least one channel and to the outlet taking with it any dispersed phase that is forced through the pores of the membrane. FIG. 21 is a simulation using a pressure of 0.25 PSI showing the deflection (not to scale) of the membrane of FIGS. 20A-B when the membrane is used for crossflow membrane emulsification. As shown in FIG. 21, the membrane deflection is significantly less than the circular membrane of FIGS. 18A-B. In addition, FIG. 22 shows the membrane deflection with respect to the pressure for the simulations using a stainless steel circular membrane of FIGS. 18A-B and the stainless steel membrane 4 of FIGS. 20A-20B.

The pores of the membrane can have a size of about 1-100 microns, about 5-75 microns, about 5-50 microns, about 10-50 microns, about 10-35 microns, about 10-20 microns, or about 15-25 microns. In some embodiments, the plurality of pores are uniformly sized. In some embodiments, the membrane can have at least about 1,000 pores, about 3,000 pores, about 5,000 pores, about 10,000 pores, about 12,500 pores, about 15,000 pores, or about 20,000 pores. In addition, the membrane can have about 1,000-20,000 pores, about 3,000-20,000 pores about 5,000-15,000 pores, about 10,000-15,000 pores, or about 12,500 pores. In some embodiments, these pores can be laser drilled in the membrane.

The regions of the membrane that include the pores are the active membrane. In some embodiments, these regions of the active membrane can have a pore density of at most about 1 pore per 225 square microns, about 1 pore per 250 square microns, about 1 pore per 275 square microns, about 1 pore per 300 square microns, about 1 pore per 350 square microns, about 1 pore per 400 square microns, about 1 pore per 450 square microns, about 1 pore per 500 square microns, about 1 pore per 550 square microns, about 1 pore per 600 square microns, about 1 pore per 650 square microns, about 1 pore per 700 square microns, about 1 pore per 750 square microns, about 1 pore per 800 square microns, about 1 pore per 850 square microns, about 1 pore per 900 square microns, about 1 pore per 950 square microns, about 1 pore per 1000 square microns, about 1 pore per 2000 square microns, about 1 pore per 5000 square microns, about 1 pore per 10000 square microns, about 1 pore per 15000 square microns, about 1 pore per 20000 square microns, or about 1 pore per 25000 square microns.

The membrane can be made out of stainless steel, tantalum, tungsten, molybdenum, manganese, tin, zinc, or an alloy thereof. In addition, the membrane can be made out of porous glass or a ceramic material. For example, the membrane can include fused silica capillaries. In some embodiments, the membrane is a hydrophilic membrane. In some embodiments, the membrane can be made out of a rigid material.

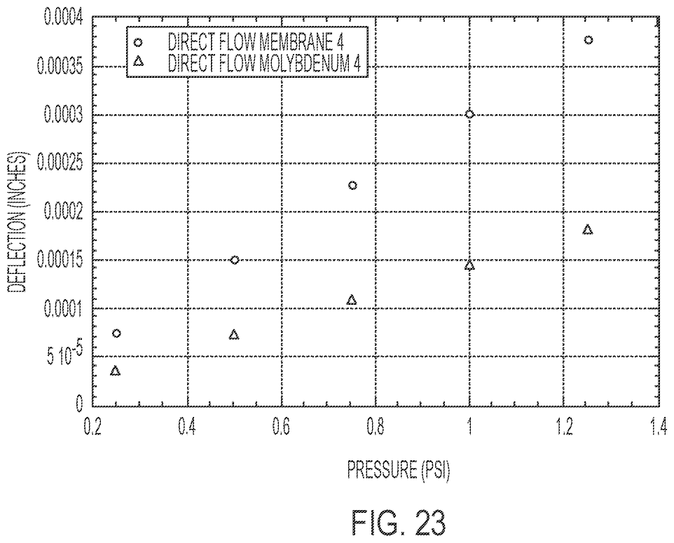

Applicants have discovered that the membrane material can impact the deflection the membrane experiences from the pressure differential between the dispersed phase (higher pressure) and the continuous phase (lower pressure). FIG. 23 shows the membrane deflection with respect to the pressure for simulations when the membrane is used for crossflow membrane emulsification using a membrane 4 made out of stainless steel and a membrane 4 made out of molybdenum. As shown in FIG. 23, the deflection is much less for a membrane made from molybdenum than a membrane made from stainless steel.

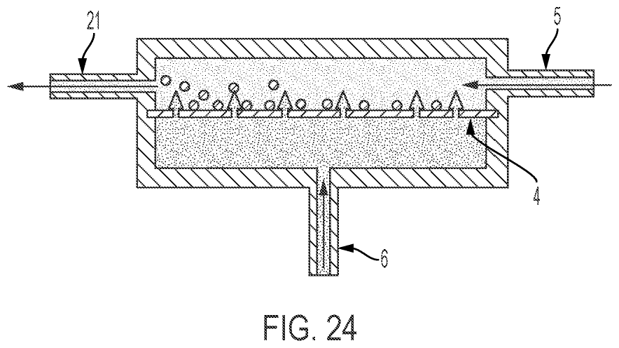

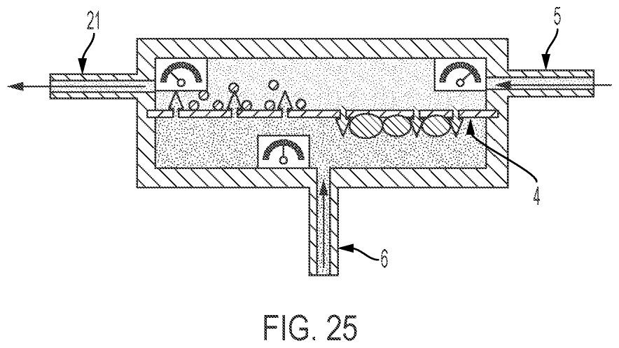

Besides membrane deflection, Applicants discovered that the pressure inside the device can cause other potential issues. As explained above and shown in FIG. 24, the dispersed phase is forced through membrane 4 to produce microspheres that are swept away by the shear cross flow of the continuous phase. However, there is a pressure gradient in the continuous phase region that is induced by the cross flow. That is, the pressure in the continuous phase space decreases from right to left as shown in FIG. 24. Because the height of the continuous phase volume can be small (to attain high shear rates with little volumetric flow), the pressure gradient can be substantial. As such, the pressure on the continuous phase side of the membrane may readily exceed the pressure on the dispersed phase side of the membrane towards the continuous phase inlet side (right side in FIG. 25) of the device as shown in FIG. 25. Accordingly, the device and in particular the shape of the continuous phase flow channel should be designed such that pressure drop across the length of the membrane in the continuous phase is smaller than the average pressure difference between the dispersed phase side of the membrane and the continuous phase side of the membrane.

Because the right-to-left flow of the continuous phase establishes a pressure gradient (as depicted by the cartoon pressure gauges in FIG. 25) and the dispersed phase has a modest pressure that can be approximately uniform because of slow flow (as depicted by the cartoon pressure gauge in FIG. 25), there can be retrograde flow of the continuous phase across the membrane into the dispersed phase as depicted by the flow arrows across the membrane in FIG. 25. Retrograde flow of the continuous phase across the membrane into the dispersed phase can accumulate in the dispersed phase side of the membrane. This retrograde flow may occur even though the dispersed phase is delivered by a positive displacement pump and there is net outflow of dispersed phase into the continuous phase.

The pressure gradient in the continuous phase side of the membrane can cause two problems. First, the pressure gradient can produce different microsphere extrusion rates along the membrane, with far more particle production towards the outlet side of the device (left side in FIG. 25). This can result in different particle size distributions for each section of the membrane. Second, is the retrograde flow of the continuous phase through the membrane into the dispersed phase towards the continuous phase inlet side (right side as shown in FIG. 25) of the device. Contamination of the dispersed phase with the continuous phase can cause portions of the dispersed phase to precipitate into sheets and/or globs that can plug the pores of the membrane. Furthermore, the dispersed phase that passes through the membrane towards the continuous phase inlet side (right side in FIG. 25) of the device exits on the outlet side of the device (left side in FIG. 25) since the dispersed phase is supplied with a positive displacement pump. This can result in high flow rate jets from the dispersed phase side of the membrane into the continuous phase side of the membrane towards the outlet side of the device (left side in FIG. 25).

Without wishing to be bound by theory, the pressure gradient on the continuous phase side of the membrane can be estimated by a laminar flow between two infinite plates in the following Equation 1:

$$G = \frac{12 Q μ}{{d}^{3}}$$

G is the pressure gradient along the membrane; Q is the flow rate per unit width of the cavity (i.e., total flow divided by the width), d is the height (distance between parallel plates), and μ is the dynamic viscosity. From Equation 1, the pressure gradient can be decreased by decreasing the flow rate or by increasing the distance between the two plates. For example, if the flow rate is held constant, but the gap between the plates is doubled, the pressure gradient decreases by a factor of 8 while the shear flow decreases by only a factor of 2. Thus, if lower shear rates are acceptable, there can be a substantial advantage to increasing the gap in terms of lowering the pressure gradient.

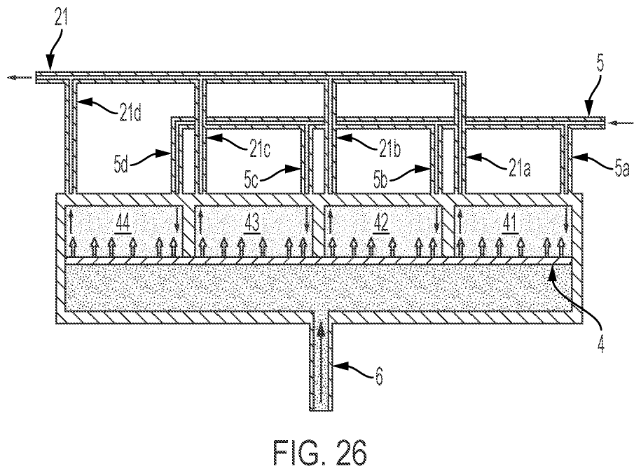

Besides decreasing the flow rate of the continuous phase or increasing the height of the continuous phase flow channel, Applicants discovered additional ways to overcome the problems associated with the pressure gradient on the continuous phase side of the membrane. The first can be to have a plurality of continuous phase inlets that each supply, in parallel, a comparatively small region of the microporous membrane and thus a small pressure drop as shown in FIG. 26. As shown in FIG. 26, a plurality of continuous phase inlets (e.g. 5a, 5b, 5c, 5d) and a plurality of outlets (e.g., 21a, 21b, 21c, 21d), in parallel, along the length of the membrane can reduce the impact of the pressure drop by making each zone (e.g., 41, 42, 43, 44) small enough that the pressure drop is not particularly consequential across each feed zone. In some embodiments, the length of the membrane can be divided into as many zones as necessary to minimize the impact of the pressure gradient. Furthermore, the plurality of continuous phase inlets and the plurality of outlets can be connected to respective manifolds or a main continuous phase inlet 5 and a main outlet 21 and thus the pressure drop across each section can be the same. These manifold dimensions can be designed in an appropriate geometry to minimize any pressure difference between sections.

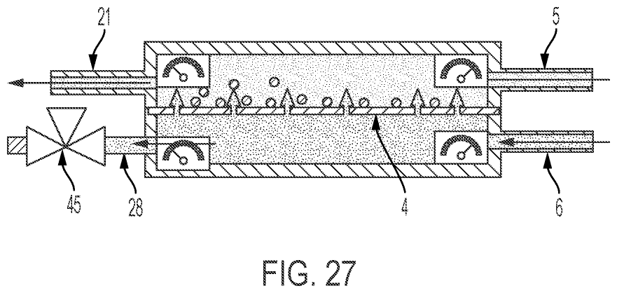

A second way to overcome the problems associated with the pressure gradient on the continuous phase side of the membrane is to introduce an equivalent pressure gradient on the dispersed phase side of the membrane such that the pressure differential is constant all along the membrane as shown in FIG. 27. As shown in FIG. 27, the dispersed phase can flow in the same direction as the continuous phase over the membrane. By adjusting the flow rate of the dispersed phase (which can be dependent on the dimensions of the dispersed phase cavity), it is possible to establish a pressure gradient that roughly matches the pressure gradient on the continuous phase side of the membrane, thus maintaining an approximately constant pressure differential from the dispersed phase to the continuous phase along the length of the membrane. A constriction 45 on the dispersed phase outlet, for example, a partially closed valve, may be used to increase the pressure of the dispersed phase to a desired value above that of the continuous phase. The constriction can adjust the net pressure between the continuous phase side of the membrane and the dispersed phase side of the membrane.

The appropriate flow rate for the dispersed phase can be determined by monitoring the pressure on both sides of the membrane at several points along the parallel flow paths and adjusting the relative flow rates to attain a matching pressure gradient in both cavities. Alternatively, approximate values for the appropriate flow rates can be determined by finite element fluid dynamic modeling or, in the case of laminar flow, by an analytical relationship. Without wishing to be bound by any theory, Applicants believe that if the continuous phase (“CP”) flow and the dispersed phase (“DP”) flow are laminar and adequately described by flow between two infinite plates (i.e., edge effects can be ignored), then an approximately matching pressure gradient may be attained under the conditions of the following Equation 2:

$$\frac{{Q}_{D P}}{{Q}_{C P}} = {( \frac{{d}_{D P}}{{d}_{C P}} )}^{3} \frac{{μ}_{C P}}{{μ}_{D P}}$$

The parameters in Equation 2 are the same as in Equation 1 except for both the continuous phase (CP) and the dispersed phase (DP). Without wishing to be bound by theory, in the approximation of infinite parallel plates (i.e., ignoring edge effects and flow through the membrane) and assuming laminar flow, the pressure gradient in the continuous phase flow (CP) and the dispersed phase flow (DP) may be equal provided the conditions in Equation 1 are met.

A third approach to achieve an appropriate pressure gradient is to design the continuous phase channels such that changes in continuous phase velocity result in corresponding pressure changes (predicted by Bernoulli's equation) that roughly balance the pressure drop induced by viscous flow. Such changes in velocity can result in changes in shear along the length of the membrane, but changes in shear rate may be tolerable so long as they do not cause unacceptable changes in the size distribution of the microparticles produced by the devices disclosed herein. Applicants discovered a comparatively small effect on particle size distributions over several-fold changes in continuous phase flow rates, and corresponding shear rates. Thus, it can be acceptable to have a variable shear rate along the length of the membrane in favor of a continuous pressure differential between the dispersed phase and the continuous phase along the length.

The pressure along the continuous phase flow path can be given by Bernoulli's equation, Equation 3:

$$P + \frac{1}{2} ρ {V}^{2} = C or \frac{d P}{d x} = - ρ V \frac{d V}{d x}$$

P is pressure; V is velocity; ρ is density, and C is a constant. To offset the pressure drop due to viscous drag using Equation 1, the following Equation 4 is obtained:

$$\frac{- 12 Q μ}{{d}^{3}} = - ρ V \frac{d V}{d x}$$

As such, the pressure drop can be offset by increasing the height of the continuous phase channel in the flow direction from the continuous phase inlet to the outlet.

By substituting Q′ for the total flow divided by the height of the continuous phase channel (i.e., Q′=Q×starting width/height) and V=Q′/width, the following Equation 5 is obtained:

$$\frac{dw}{dx} = \frac{12 {Q}^{′} μ w}{ρ {d}^{2}}$$

As such, Equation 5 provides the increase in width of the continuous phase channel in the flow direction from the continuous phase inlet to outlet required to offset viscous drag (assuming laminar flow), where w is the width of the channel. Equation 5 may be integrated to obtain an expression for the width of the channel as a function of distance, as shown in the following Equation 6:

$$ln ( \frac{w ( x )}{{w}_{0}} ) = \frac{12 {Q}^{′} μ x}{ρ {d}^{2}}$$

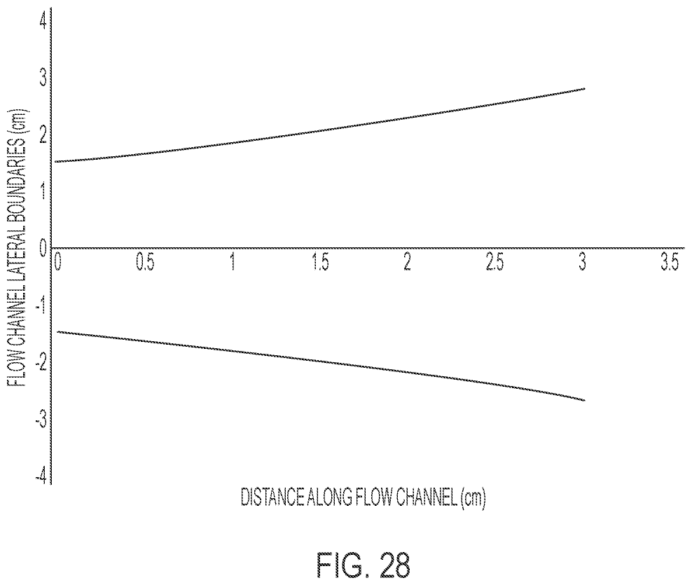

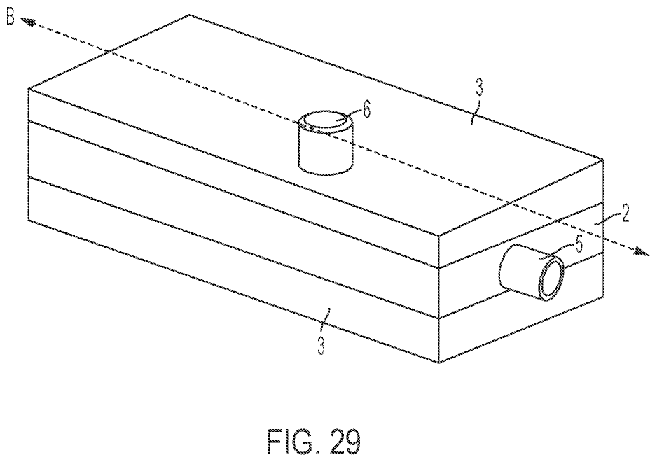

Equation 6 provides a relationship for the width of the continuous phase channel such that the pressure drop by viscous drag is approximately offset by increased pressure induced by widening the channel. Although a curve described by Equation 6 is complex, such a path may be generated with modern CNC equipment. An example of such a channel width generated is shown in FIG. 28. In FIG. 28, flow would be from left to right and the conditions modeled were water at a volumetric flow rate of 100 mL/min, a channel height of 1 cm, and an initial channel width of 3 cm. The curvature of the channel increases with flow rate, but for modest flow rates, such as the example here, a linear widening of the channel may suffice to maintain an approximately constant pressure differential across the membrane between the dispersed phase and the continuous phase. In the example above, the shear rate may decrease by a factor of 1.8 along the path length. As such, the overall length of the channel may be limited by changes in the particle distribution size induced by the changes in the shear flow.

The principle of widening the channel may be applied to both the height and width of the continuous phase channel. Thus, for higher rates of flow, the curvature of the channel boundaries may be mitigated by increasing the height and/or width of the channel. Accordingly, the cross section of the continuous phase channel changes as fluid flows from the continuous phase inlet to the outlet such that the continuous phase pressure gradient is reduced or eliminated.

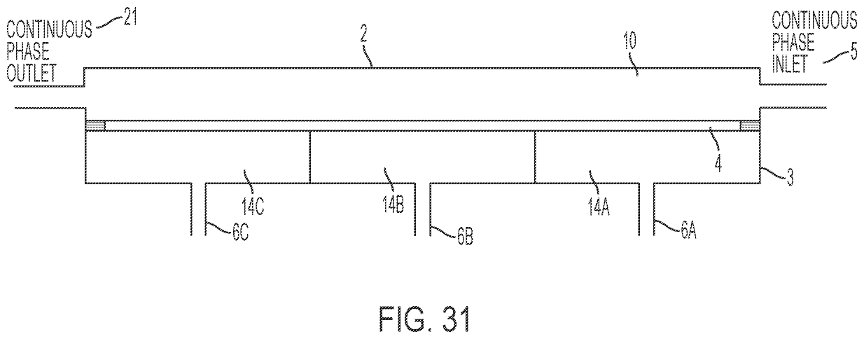

In some embodiments, membrane emulsification devices may be used in series. In some such embodiments, the continuous phase outlet of one device can be connected to the continuous phase inlet of another device. The dispersed phase for each section can be fed by a positive displacement pump. In other embodiments, the devices could be integrated into a single unit with multiple dispersed phase feeds. Such an embodiment is shown in FIG. 31. FIG. 31 depicts the dispersed phase cavity divided into a manifold of cavities 14A, 14B, and 14C. Each of the cavities has its own dispersed phase inlets 6A, 6B, and 6C. These individual dispersed phase inlets can be supplied by a separate positive displacement pump. Each sequential dispersed phase segment can have a lower pressure because the positive displacement pump will be working against a successively low continuous phase pressure. In some embodiments, each of the cavities can be connected to the next cavity by a constriction that can induce a pressure drop sequentially from one cavity to the next that matches the corresponding pressure drop on the continuous phase side of the membrane.

To have a long continuous phase channel and commensurate pressure drop along the continuous phase channel, it may be necessary that the dispersed phase to continuous phase pressure drop be larger than the pressure drop along the continuous phase channel. To do this, it is possible to use a viscous dispersed phase and a small pore size for a given flow rate. According to theory, the dispersed phase to continuous phase pressure drop depends on the viscosity to the first power but the pore size to the fourth power (at a given flow rate). Thus, decreasing the pore size by a factor of two can produce a roughly sixteen times higher pressure in the dispersed phase side. As such, a much longer channel is possible.

The membrane used in the device can come in a variety of shapes and sizes. For example, the length of the membrane can be about 60-100 mm, about 70-95 mm, about 75-95 mm, about 80-90 mm, or about 85 mm. In some embodiments, the length of the membrane can be greater than about 100 mm, about 500 mm, or about 1000 mm. The width of the membrane can be about 10-50 mm, about 15-40 mm, about 20-40 mm, about 25-35 mm, or about 33 mm. In some embodiments, the width of the membrane can be greater than about 50 mm, about 100 mm, about 500 mm, or about 1000 mm. The thickness of the membrane can be about 0.01-1 mm, about 0.05-0.5 mm, about 0.08-0.2 mm, or about 0.1 mm. Furthermore, the membrane may be free of all dents, creases, and burrs.

The membrane can also have alignment holes 17. These alignment holes can be for mounting the membrane on the dispersed phase plate. For example, the dispersed phase plate can have alignment pegs 18 in notch 19 on a surface of the dispersed phase plate facing the membrane as shown in FIGS. 2, 12-13, and 41. The alignment pegs can fit into the alignment holes of the membrane and the membrane can fit in the notch of the dispersed phase plate. The membrane can fit in the notch of the dispersed phase plate such that the membrane is flush with the surface of the dispersed phase plate outside of the notch. In some embodiments, the continuous phase plate can also include alignment holes 30 for the alignment pegs 18 as shown in FIG. 14. As such, the alignment pegs can fit into the alignment holes of the membrane and then into the alignment holes of the continuous phase plate. Accordingly, the continuous phase plate can compress against the membrane and the dispersed phase plate so as to create a fluid tight connection.

In some embodiments, the notch and/or alignment pegs can be in a surface of the continuous phase plate facing the membrane and the dispersed phase plate can include the alignment holes. The alignment pegs can fit into the alignment holes of the membrane and the membrane can fit in the notch of the continuous phase plate. The membrane can fit in the notch of the continuous phase plate such that the membrane is flush with the surface of the continuous phase plate outside of the notch. Accordingly, the dispersed phase plate can compress against the membrane and the continuous phase plate so as to create a fluid tight connection.

In some embodiments, the notch can include the cavity and/or a gasket trough. For example, FIGS. 12-13 and 41 illustrate gasket trough 29 for gasket 27. The gasket trough of the dispersed phase plate can surround the cavity. The combination of the gasket trough of the dispersed phase plate and the gasket can fill the space between the membrane and the dispersed phase plate to prevent any of the dispersed phase from leaking outside of the cavity. As such, the dispersed phase can travel through the dispersed phase inlet, through the cavity, through the pores in the membrane, through the at least one channel with the continuous phase, and then out the outlet. In some embodiments, some of the dispersed phase does not travel through the pores in the membrane. In such instances, some of the dispersed phase can travel through the dispersed phase inlet, through the cavity, and then out the dispersed phase outlet.

In some embodiments, the continuous phase plate can include gasket trough 29 as shown in FIG. 14. The gasket trough of the continuous phase plate can surround the continuous phase flow path from inlet to outlet. The combination of the gasket trough of the continuous flow path and the gasket can fill the space between the membrane and the continuous phase plate to prevent any of the continuous phase (or continuous phase with dispersed phase microspheres) from leaking outside of the channels and continuous phase flow path. As such, the continuous phase can travel through the continuous phase inlet, through the at least one channel, and then out the outlet

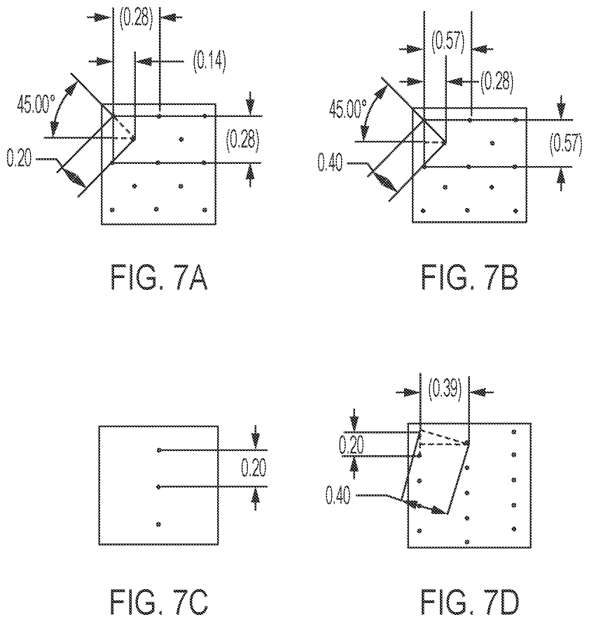

The pore configuration in the at least one region of the membrane can vary. FIGS. 7A-7D provide various examples for the pore configuration in the at least one region of the membrane. As shown in FIGS. 7A, 7B, and 7D, the columns of pores can be shifted compared to the other pores. In contrast to FIGS. 7A, 7B, and 7D, the columns of pores can be identical throughout the length of the at least one region as shown in FIG. 7C and illustrated in FIGS. 2 and 6. Furthermore, the pores in one column of pores in the at least one region of the membrane can be separated by about 0.1-1 mm, about 0.15-0.75 mm, about 0.2-0.57 mm, or about 0.2-0.28 mm. In addition, one column of pores can be separated from another column of pores by about 0.1-1.1 mm, about 0.14-0.57 mm, about 0.28-0.57 mm, or about 0.39-0.57 mm. In some embodiments, the pores can be in an anisotropic fashion (when pores are further spaced along the axis of the continuous flow than across it).

As explained above, forcing the dispersed phase through the plurality of pores into the continuous phase can form a plurality of dispersed phase microspheres. The device disclosed herein is capable of producing microspheres with a narrow size distribution. Specifically, the median diameter of the plurality of microspheres can be about 5-100 microns, about 10-50 microns, or about 20-40 microns. In some embodiments, at least about 50%, about 60%, about 70%, about 80%, about 85%, about 90%, about 95%, or about 98% of the plurality of microspheres can have a diameter within 5, 10, 15, or 20 microns above or below the median diameter. In some embodiments, the microspheres have a bimodal distribution wherein a first mode occurs at a diameter of less than about 5, 10, 15, or 20 microns and the second mode occurs at a diameter of 5, 10, 15, 20, 25, 30, 35, or 40 microns or greater where at least about 50%, about 60%, about 70%, about 80%, about 85%, about 90%, about 95%, or about 98% of the plurality of microspheres can have a diameter within 5, 10, 15, or 20 microns above or below the median diameter.

In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 22-36 μm in diameter. In some embodiments, the present disclosure provides a plurality of microspheres, where at least 90-95% of the microspheres are 22-36 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 26-34 μm in diameter. In some embodiments, the present disclosure provides a plurality of microspheres, where at least 60-70% of the microspheres are 26-34 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 20-40 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 28-32 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 22-34 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 26-36 μm in diameter.

In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 6% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 6% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 5% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 5% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 10% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 10% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 15% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 15% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 20% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 20% of the median diameter of the plurality of microspheres.

In some embodiments, the coefficient of variation of a size distribution of the plurality of microspheres can be less than about 40%, about 30%, about 25%, about 20%, about 15%, or about 10%. In some embodiments, the coefficient of variation of a size distribution of the plurality of microspheres can be about 1-30%, about 5-25%, or about 10-20%. The coefficient of variation of a size distribution can be calculated by performing image recognition on light microscopy pictures (Cell Profiler for image analysis). The coefficient of variation can be calculated using the standard error of the population (diameter) divided by the average diameter and multiply by 100.

As the dispersed phase is forced through the pores of the membrane, the continuous phase can exert a shear force at a shear rate at the membrane. The shear rate at the membrane can be at most about 25,000 s⁻¹, about 19,000 s⁻¹, about 15,000 s⁻¹, about 10,000 s⁻¹, about 9,000 s⁻¹, or about 5,000 s⁻¹. In some embodiments, the shear rate at the membrane can be about 1,000-20,000 s⁻¹, 2,000-20,000 s⁻¹, about 5,000-19,000 s⁻¹, about 5,000-15,000 s⁻¹, or about 5,000-10,000 s⁻¹. In some embodiments, the shear rate at the membrane can be about 1,000-10,000 s⁻¹, about 2,000-8,000 s⁻¹, about 3,000-6,000 s⁻¹, or about 4,000-5,000 s⁻¹. The shear rate at the membrane can be calculated by using simple shear defined by the gradient of velocity defined as the average flow divided by the distance.

After the microspheres are washed from the membrane by the continuous phase flow, the continuous phase and the microspheres carried by the continuous phase can flow to the outlet. In some embodiments, the device may only have one outlet. In other embodiments, the device may have at least two outlets: a continuous phase outlet; and a dispersed phase outlet. In some embodiments, the outlet is on an outer surface of the continuous phase plate. The outlet does not have to be on an outer surface. Instead, the outlet can be on any one of the side surfaces of the continuous phase plate. In some embodiments, the outlet can be on an outer surface that is not on the same plane as the top most outer surface of the continuous phase plate as shown in FIG. 32. As shown in FIG. 32, the outlet can be on an outer surface of the continuous phase plate that slants toward the dispersed phase plate. In addition, this slanting surface can slant away from the center of the continuous phase plate. In addition, the outlet can be closer to one end of the continuous phase plate than the middle of the plate, as depicted in FIG. 1 as outlet 21. In some embodiments, the outlet can be on the same side of the membrane as the continuous phase flow through the device. In some embodiments, the outlet can be a separate structural part attached to the continuous phase plate and/or the dispersed phase plate that is fluidly connected with the at least one channel.

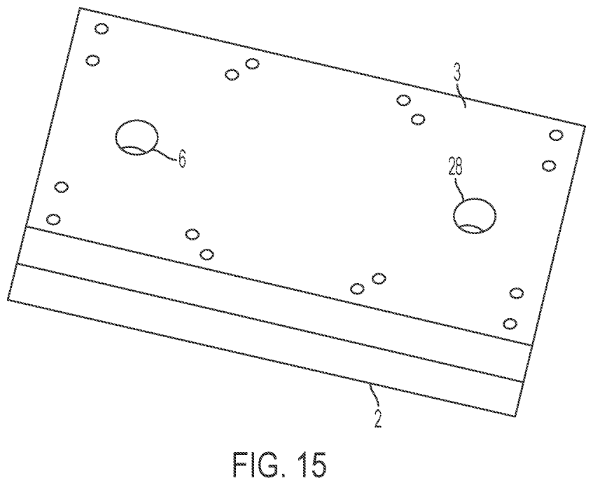

In some embodiments, the dispersed phase plate can include a dispersed phase outlet. For example, FIG. 15 illustrates dispersed phase outlet 28. As such, dispersed phase from the dispersed phase inlet that does not travel through the pores of the membrane can be removed from the device through the dispersed phase outlet. In some embodiments, the dispersed phase outlet is on an outer surface of the dispersed phase plate. The dispersed phase outlet does not have to be on the outer surface. Instead, the dispersed phase outlet can be on any one of the side surfaces of the dispersed phase plate. In some embodiments, the dispersed phase outlet can be closer to one end of the dispersed phase plate than the middle of the plate. In some embodiments, the dispersed phase outlet is centered on the dispersed phase plate. In some embodiments, the dispersed phase outlet can be the bleed valve discussed above.

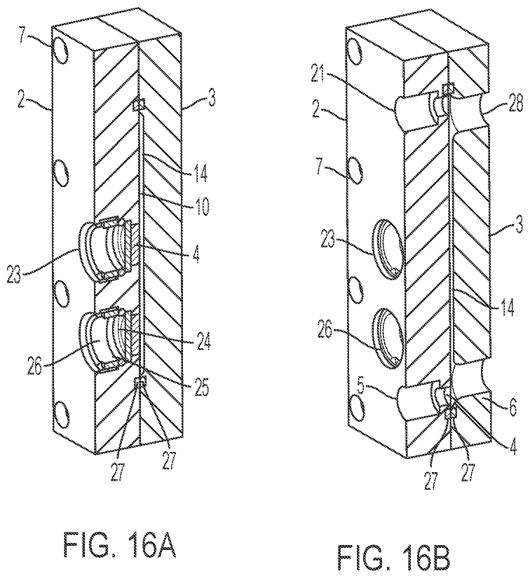

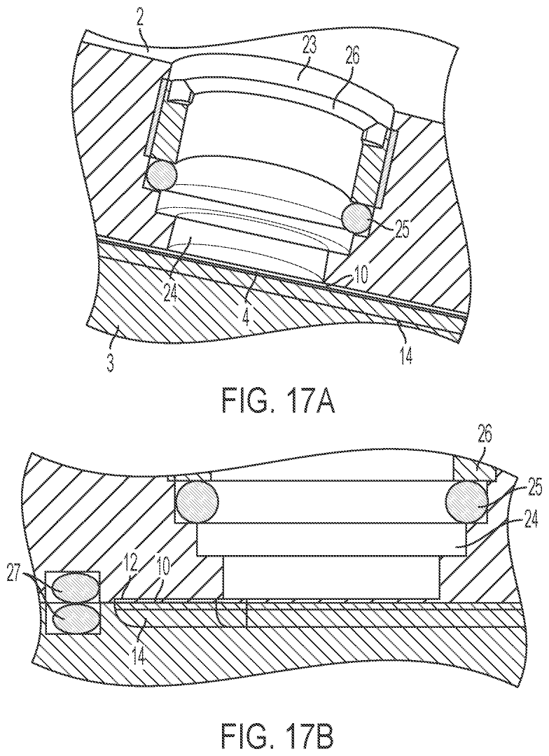

FIG. 16A is a lengthwise cross section of the device through axis C shown in FIG. 10. FIGS. 17A and 17B are a close-up of the cross section through at least one viewing window 23 of FIG. 16A. As shown in FIGS. 16A and 17A-17B, the at least one viewing window can form a part of the at least one channel. As such, the at least one groove can include at least a portion of the at least one viewing window. Specifically, FIG. 17B illustrates the at least one channel 10 formed from at least one groove 12, membrane 4, and the at least one viewing window 23 (more specifically, the at least one transparent window 24 of at least one viewing window 23). The at least one transparent window can fit in the at least one viewing window such that the at least one transparent window is flush with the surface of the at least one groove of the continuous phase plate as shown in FIG. 17B. In addition, the at least one transparent window can fit in the at least one viewing window so as to create a fluid tight connection.

FIG. 16B does not include a channel in the cross-section of the device because channels 10 and 11 are not directly on axis D of the device. Although FIGS. 10-17B have illustrated the continuous phase and the dispersed phase flowing in the same direction into and out of the device, the continuous phase and the dispersed phase can have a counter current arrangement such that at least one of the inlets and the corresponding outlet for one of the plates are switched.

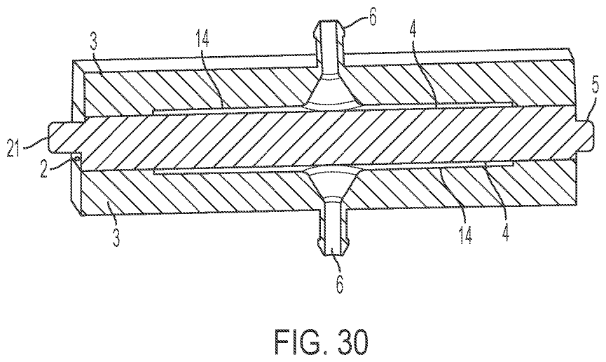

In some embodiments, the membrane emulsification device can include two dispersed phase plates 3 as shown in FIG. 29 that sandwich continuous phase plate 2. In such an embodiment, the membrane emulsification device can include more than one membrane. For example, a cross section (FIG. 30) along axis B in FIG. 29 shows that the dispersed phase can enter the continuous phase through a top and bottom membrane 4. As such, the continuous phase can flow in a direction that is perpendicular to dispersed phase flow through the plurality of pores in both the top and bottom membranes 4.

### Dispersed Phase and Continuous Phase Flows

The continuous phase can include an aqueous solvent and the dispersed phase can include an organic solvent. The continuous phase can also include a surfactant. A variety of surfactants are known in the art and can be selected by one of skill in the art. In some embodiments, the surfactant is selected from polysorbate 20 or polysorbate 80 (e.g., of the TWEEN® series), poloxamer (e.g., of the PLURONIC® series; BASF), and polyvinyl alcohol (PVA). In some embodiments, the concentration of surfactant in the continuous phase is from 0.05% to 2% (w/w). In some embodiments, the concentration of surfactant in the continuous phase is at least about any of the following concentrations (in percentage, w/w): 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2. In some embodiments, the concentration of surfactant in the continuous phase is less than about any of the following concentrations (in percentage, w/w): 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1. That is, the concentration of surfactant in the continuous phase can be any concentration in a range having an upper limit of about 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w), and an independently selected lower limit of about 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2% (w/w), wherein the upper limit is greater than the lower limit. In some embodiments, the concentration of surfactant in the continuous phase is about 0.5-1.5% (w/w). In some embodiments, the concentration of surfactant in the continuous phase is about 0.5-1% (w/w). In some embodiments, the aqueous solvent comprises water.

The dispersed phase can also include a water insoluble polymer. The water insoluble polymer can dissolve in the organic solvent. Examples of water insoluble polymers include, but are not limited to, poly(lactic-co-glycolic acid) (PLGA) including PLGA that is ester capped, polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer, or combinations thereof. In some embodiments, the organic solvent comprises ethanol, methanol, propanol, dichloromethane, chloroform, ethyl acetate, butyl acetate, methyl ethyl ketone, or mixtures thereof.

In some embodiments, the dispersed phase can include a therapeutic compound or a pharmaceutically acceptable salt thereof. In some embodiments, the dispersed phase can also include a polyol.

In some embodiments, the dispersed phase comprises one or more polymers at a concentration of at least about 75 mg/mL, at least about 100 mg/mL, at least about 125 mg/mL, at least about 150 mg/mL, at least about 160 mg/mL, at least about 170 mg/mL, at least about 180 mg/mL, at least about 190 mg/mL, at least about 200 mg/mL, at least about 225 mg/mL, at least about 250 mg/mL, at least about 275 mg/mL, or at least about 300 mg/mL. In some embodiments, the dispersed phase comprises a therapeutic compound or salt at a concentration of at least about 5 mg/mL, at least about 10 mg/mL, at least about 20 mg/mL, at least about 30 mg/mL, or at least about 40 mg/mL. In some embodiments, the dispersed phase comprises the one or more polymers at a concentration of at least about 200 mg/mL and the therapeutic compound or salt at a concentration of at least about 20 mg/mL.

In some embodiments, a polymer of the present disclosure comprises at least one anionic terminus. In some embodiments, a polymer of the present disclosure comprises at least one acid terminus. In certain embodiments, a polymer of the present disclosure comprises at least one carboxylic acid terminus. Exemplary polymers include, without limitation, those comprising poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

Exemplary polymers can also include those prepared from biocompatible and biodegradable polymers, such as linear polyesters, branched polyesters which are linear chains radiating from a polyol moiety, e.g. glucose. Other esters are those of polylactic acid, polyglycolic acid, polyhydroxybutyric acid, polycaprolactone, polyalkylene oxalate, polyalkylene glycol esters of acids of the Kreb's cycle, e.g. citric acid cycle and the like and copolymers thereof. The linear polyesters may be prepared from the alphahydroxy carboxylic acids, e.g. lactic acid. and glycolic acid, by the condensation of the lactone dimers, see for example U.S. Pat. No. 3,773,919.

The branched polyesters may be prepared using polyhydroxy compounds e.g. polyol e.g. glucose or mannitol as the initiator. These esters of a polyol are known and described in GB 2,145,422 B. The polyol contains at least 3 hydroxy groups and has a molecular weight of up to 20 kD, with at least 1, at least 2, e.g. as a mean 3 of the hydroxy groups of the polyol being in the form of ester groups, which contain poly-lactide or co-poly-lactide chains. Typically 0.2% glucose is used to initiate polymerization. The structure of the branched polyesters may be star shaped. The polyester chains in the linear and star polymer compounds optionally used according to the present disclosure are copolymers of the alpha carboxylic acid moieties, lactic acid and glycolic acid, or of the lactone dimers. The molar ratios of lactide:glycolide is from about 5:25 to 25:75, e.g. 60:40 to 40:60, with from 55:45 to 45:55, e.g. 55:45 to 50:50. The star polymers may be prepared by reacting a polyol with a lactide and optionally also a glycolide at an elevated temperature in the presence of a catalyst, which makes a ring opening polymerization feasible. Alternatively, the star polymers may be prepared by reacting a polycarboxylic acid (e.g., maleic acid) with hydroxyl-containing monomers in a polymerization reaction.

In some embodiments, a polymer of the present disclosure comprises a molecular weight less than or equal to 17 kD. In some embodiments, a molecular weight refers to the average molecular weight of a polymer species. In some embodiments, a molecular weight refers to the minimum or maximum molecular weight of a polymer species. For example, RESOMER® RG 502H (Evonik Industries) has a molecular weight of 7 kD-17 kD, and RESOMER® RG 503H (Evonik Industries) has a molecular weight of 24 kD-38 kD. In some embodiments, a polymer of the present disclosure comprises a maximum molecular weight less than or equal to 17 kD. In some embodiments, a polymer of the present disclosure comprises a minimum molecular weight less than or equal to 7 kD. In some embodiments, a polymer of the present disclosure comprises a maximum molecular weight less than or equal to 38 kD. In some embodiments, a polymer of the present disclosure comprises a minimum molecular weight less than or equal to 24 kD.

In some embodiments, a microsphere of the present disclosure comprises (or is made with a dispersed phase comprising) more than one polymer, e.g., 2, 3, 4, 5, or more polymers. In some embodiments, at least one of the polymers has a pI at least 1.5 units lower than the pI of the therapeutic compound or salt. In some embodiments, at least one of the polymers comprises one or more anionic termini. In some embodiments, a first of the multiple polymers has a lower molecular weight than a second of the multiple polymers. In some embodiments, a first of the multiple polymers has a lower molecular weight (e.g., average, minimum, or maximum molecular weight) by at least 10 kD than a second of the multiple polymers. In some embodiments, the molecular weight (e.g., average, minimum, or maximum molecular weight) of the first polymer is less than or equal to 17 kD. In some embodiments, the molecular weight (e.g., average, minimum, or maximum molecular weight) of the first polymer is less than or equal to 17 kD, and the molecular weight (e.g., average, minimum, or maximum molecular weight) of the second polymer is at least 24 kD. In some embodiments, the first polymer has one or more anionic termini, and the second polymer does not.

In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer; and the second polymer comprises a polymer independently selected from poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer; and the second polymer comprises the same polymer (but a species thereof having a different molecular weight than the first polymer) of poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first and second polymers represent different species of PLGA. In some embodiments, the first and second polymers represent different species of PLGA that both comprise a carboxylic acid terminus. In some embodiments, the first and second polymers are PLGA species having a difference in minimum molecular weight of at least about 7 kD, at least about 10 kD, at least about 17 kD, or at least about 20 kD. In some embodiments, the first and second polymers are PLGA species having a difference in maximum molecular weight of at least about 7 kD, at least about 10 kD, at least about 17 kD, or at least about 20 kD. In some embodiments, the first and the second polymers both comprise PLGA, and the molecular weight (e.g., average, minimum, or maximum molecular weight) of the first polymer is at least 10 kD lower than the molecular weight of the second polymer. In some embodiments, a microsphere of the present disclosure comprises (or is made with a dispersed phase comprising) PLGA having a molecular weight of 7 kD-17 kD, and PLGA having a molecular weight of 24 kD-38 kD.

In some embodiments, a microsphere of the present disclosure comprises (or is made with a dispersed phase comprising) the first and the second polymer at a ratio of between about 20:80 and about 80:20 (first polymer:second polymer). In some embodiments, a microsphere of the present disclosure comprises (or is made with a dispersed phase comprising) the first and the second polymer at a ratio of greater than 20:80, 25:75, 30:70, 35:65, 40:60, 45:55, 50:50, 55:45, 60:40, 65:35, 70:30, or 75:25 (first polymer:second polymer). In some embodiments, a microsphere of the present disclosure comprises (or is made with a dispersed phase comprising) the first and the second polymer at a ratio of less than 80:20, 75:25, 70:30, 65:35, 60:40, 55:45, 50:50, 45:55, 40:60, 35:65, 30:70, or 25:75 (first polymer:second polymer). That is, a microsphere of the present disclosure comprises (or is made with a dispersed phase comprising) the first and the second polymer at a ratio having an upper limit of 80:20, 75:25, 70:30, 65:35, 60:40, 55:45, 50:50, 45:55, 40:60, 35:65, 30:70, or 25:75 and an independently selected lower limit of 20:80, 25:75, 30:70, 35:65, 40:60, 45:55, 50:50, 55:45, 60:40, 65:35, 70:30, or 75:25 (first polymer:second polymer), wherein the upper limit is greater than the lower limit. In some embodiments, a microsphere of the present disclosure comprises (or is made with a dispersed phase comprising) the first and the second polymer at a ratio of 20:80, 25:75, 30:70, 35:65, 40:60, 45:55, 50:50, 55:45, 60:40, 65:35, 70:30, 75:25, or 80:20 (first polymer:second polymer). In certain embodiments, a microsphere of the present disclosure comprises (or is made with a dispersed phase comprising) the first and the second polymer at a ratio of about 75:25 (first polymer:second polymer).

In some embodiments, the dispersed phase comprises a ratio of between 150:30 to 300:10, between 200:30 and 200:20, and between 5:1 and 10:3 (first polymer:therapeutic compound or salt) by weight. In some embodiments, the dispersed phase comprises a ratio of greater than any of 5:1, 6:1, 7:1, 8:1, 9:1, or 10:1 (first polymer:therapeutic compound or salt) by weight. In some embodiments, the dispersed phase comprises a ratio of less than any of 10:3, 10:2, 10:1, 9:1, 8:1, 7:1, or 6:1 (first polymer:therapeutic compound or salt) by weight. That is, the dispersed phase can comprise any ratio in a range of ratios having an upper limit of 10:3, 10:2, 10:1, 9:1, 8:1, 7:1, or 6:1 (first polymer:therapeutic compound or salt) and an independently selected lower limit of 5:1, 6:1, 7:1, 8:1, 9:1, or 10:1 (first polymer:therapeutic compound or salt), wherein the upper limit is greater than the lower limit. In some embodiments, the dispersed phase comprises a ratio of between 10:1 and 10:3 (first polymer:therapeutic compound or salt) by weight, e.g., a ratio of 10:1, 10:2, or 10:3 (first polymer:therapeutic compound or salt) by weight.

In some embodiments, the dispersed phase comprises a therapeutic compound or salt of the present disclosure at a concentration of about 10-60 mg/mL by weight. In some embodiments, the dispersed phase comprises a therapeutic compound or salt of the present disclosure at a concentration of about 20-40 mg/mL by weight.

In some embodiments, a therapeutic compound or salt of the present disclosure comprises at least one cationic moiety.

In some embodiments, a therapeutic compound or salt of the present disclosure comprises a therapeutic peptide. In some embodiments, the therapeutic peptide comprises at least two amino-containing amino acid side chains. In some embodiments, the therapeutic peptide has a length from 6 to 40 amino acids, e.g., a length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In certain embodiments, the therapeutic peptide has a length of 8 amino acids. In some embodiments, the therapeutic peptide is cyclic. In some embodiments, the therapeutic peptide is selected from veldoreotide, somatostatin (SST-28), SST-14, lanreotide, octreotide, vapreotide, pasireotide, and pharmaceutically acceptable salts of any of the foregoing. In some embodiments, the therapeutic peptide is human growth hormone or a pharmaceutically acceptable salt thereof. In some embodiments, the therapeutic peptide is octreotide or a pharmaceutically acceptable salt thereof.

In some embodiments, the therapeutic peptide is a somatostatin analog or a pharmaceutically acceptable salt thereof. Naturally occurring somatostatin is produced by the hypothalamus as well as other organs, e.g. the gastrointestinal tract, and mediates, together with growth-hormone releasing factor (GRF), the neuroregulation of pituitary growth hormone release. In addition to inhibition of growth hormone (GH) release by the pituitary, somatostatin is a potent inhibitor of a number of systems, including central and peripheral neural, gastrointestinal and vascular smooth muscle. It also inhibits the release of insulin and glucagon. Analogs (e.g., agonist analogs) of somatostatin are thus useful in replacing natural somatostatin in its effect on regulation of physiologic functions. For exemplary descriptions of somatostatin analogs, see, e.g., U.S. Pat. No. 5,639,480. Naturally occurring somatostatin is a tetradecapeptide having the structure:

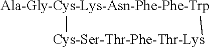

As used herein, the term “somatostatin” includes its analogues or derivatives thereof. By derivatives and analogues is understood straight-chain, bridged or cyclic polypeptides wherein one or more amino acid units have been omitted and/or replaced by one or more other amino radical(s) of and/or wherein one or more functional groups have been replaced by one or more other functional groups and/or one or more groups have been replaced by one or several other isosteric groups. In general, the term covers all modified derivatives of a biologically active peptide which exhibit a qualitatively similar effect to that of the unmodified somatostatin peptide.

The term derivative includes also the corresponding derivatives bearing a sugar residue. When somatostatins bear a sugar residue, this is can be coupled to an N-terminal amino group and/or to at least one amino group present in a peptide side chain, such as to a N-terminal amino group. Such compounds and their preparation are disclosed, e.g. in WO 88/02756. Exemplary derivatives are N.sup.α-[α-glucosyl-(1-4-deoxyfructosyl]-DPhe-Cys-Phe-DTrp-Lys-Thr-Cys-Thr-ol and N.sup.α-[β-deoxyfructosyl-DPhe-Cys-Phe-DTrp-Lys-Thr-Cys-Thr-ol, each having a bridge between the -Cys- moieties, optionally in acetate salt form and described in Examples 2 and 1 respectively of the above mentioned application.

In some embodiments, the therapeutic peptide is selected from somatostatin (SST-28), SST-14, lanreotide, octreotide, vapreotide, pasireotide, and pharmaceutically acceptable salts of any of the foregoing. Octreotide derivatives are also contemplated for use and include, without limitation, those comprising the moiety:

* *
-D-Phe-Cys-Phe-DTrp-Lys-Thr-Cys-

having a bridge between Cys residues.

The somatostatins may exist e.g. in free form, salt form or in the form of complexes thereof. Acid addition salts may be formed with e.g. organic acids, polymeric acids and inorganic acids. Acid addition salts include e.g. the hydrochloride and acetates. Complexes are e.g. formed from somatostatins on addition of inorganic substances, e.g. inorganic salts or hydroxides such as Ca- and Zn-salts and/or an addition of polymeric organic substances.

The acetate salt is an exemplary salt for such formulations, especially for microspheres leading to a reduced initial drug burst. The present disclosure also provides the pamoate salt, which is useful, particularly for implants and the process for its preparation. The pamoate may be obtained in conventional manner, e.g. by reacting embonic acid (pamoic acid) with octreotide e.g. in free base form. The reaction may be effected in a polar solvent, e.g. at or below room temperature.

In some embodiments, a therapeutic compound or salt of the present disclosure comprises a small molecule drug or compound.

In some embodiments, the therapeutic compound or salt comprises an mTOR inhibitor. The term “mTOR inhibitor” broadly encompasses multiple classes of molecules, including molecules that bind FKBP12 (e.g., first-generation mTOR inhibitors such as rapamycin and rapalogs that inhibit mTORC1), molecules that inhibit the kinase activity of mTOR (e.g., second-generation, ATP-competitive mTOR inhibitors that inhibit mTORC1 and mTORC2), molecules that bind FKBP12 and inhibit the kinase activity of mTOR (e.g., third-generation mTOR inhibitors such as RapaLinks), and dual PI3K/mTOR inhibitors (e.g., BEZ235 or LY3023414). In some embodiments, an mTOR inhibitor inhibits mTORC1, mTORC2, or both. Examples of specific mTOR inhibitors include, without limitation, tacrolimus (also known as FK506, fujimycin, PROGRAF®, PROTOPIC®, ADVAGRAF®, ENVARSUS®, and ASTAGRAF®), temsirolimus (also known as CCI-779 and TORISEL®), everolimus (also known as RAD001, ZORTRESS®, AFINITOR®, CERTICAN®, VOTUBIA®, and Evertor), rapamycin (also known as sirolimus and RAPAMUNE®), ridaforolimus (also known as AP23573, MK-8669, and deforolimus), AZD8055, Ku-0063794, PP242, PP30, Torin1, WYE-354, PI-103, BEZ235 (also known as NVP-BEZ235 and dactolisib), PKI-179 (also known as PKI-587), LY3023414, omipalisib (also known as GSK2126458 and GSK458), sapanisertib (also known as MLN0128 and INK128), OSI-027, RapaLink-land voxtalisib (also known as XL765 and SAR245409).

In some embodiments, the therapeutic compound or salt comprises a glucocorticoid, e.g., a compound that binds the glucocorticoid receptor. Examples of specific glucocorticoids include, without limitation, triamcinolone (e.g., triamcinolone acetonide), beclomethasone, betamethasone, budesonide, cortisone, hydrocortisone, methylprednisolone, prednisolone, prednisone, and dexamethasone.

In some embodiments, the therapeutic compound or salt comprises a Janus kinase (JAK) inhibitor. The term “JAK inhibitor” broadly encompasses molecules that inhibit the function of one or more JAK family kinases, such as JAK1, JAK2, JAK3, and TYK2. For example, in some embodiments, a JAK inhibitor inhibits one or more activities of JAK1; JAK2; JAK3; JAK1 and JAK2; JAK1 and JAK3; JAK3 and JAK2; TYK2 and JAK1; TYK2 and JAK2; TYK2 and JAK3; JAK1, JAK2, and JAK3; or JAK1, JAK2, TYK2, and JAK3. Examples of specific JAK inhibitors include, without limitation, ruxolitinib (also known as JAKAFI®, JAKAVI®, and INCB018424, including the phosphate and sulfate salts and S enantiomer), tofacitinib (also known as tasocitinib, CP-690550, XELJANZ® and JAKVINUS®, including (3R,4S), (3S,4R), and (3S,4S) enantiomers and the citrate salt), oclacitinib (also known as APOQUEL®, including the maleate salt), baricitinib (also known as LY3009104, INCB-28050, and OLUMIANT®, including the phosphate salt), filgotinib (also known as G-146034 and GLPG-0634), gandotinib (also known as LY-2784544), lestaurtinib (also known as CEP-701), momelotinib (also known as GS-0387 and CYT-387, including mesylate and sulfate salts), pacritinib (also known as SB1518), PF-04965842, upadacitinib (also known as ABT-494), peficitinib (also known as ASP015K and JNJ-54781532), fedratinib (also known as SAR302503 and TG101348), cucurbitacin I (also known as JSI-124), decernotinib (also known as VX-509 and VRT-831509), INCB018424, AC430, BMS-0911543, GSK2586184, VX-509, R348, AZD1480, CHZ868, PF-956980, AG490, WP-1034, JAK3 inhibitor IV (also known as ZM-39923, including the hydrochloride salt), atiprimod (including the dihydrochloride salt), FM-381, SAR20347, AZD4205, ARN4079, NIBR-3049, PRN371, PF-06651600 (including the malonate salt), JAK3i, JAK3 inhibitor 31, PF-06700841 (including the tosylate salt), NC1153, EP009, Gingerenone A, JANEX-1 (also known as WHI-P131), cercosporamide, JAK3-IN-2, PF-956980, Tyk2-IN-30, Tyk2-IN-2, JAK3-IN1, WHI-P97, TG-101209, AZ960, NVP-BSK805 (including the dihydrochloride salt), NSC 42834 (also known as Z3), FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib (also known as GSK-2586184 and GLPG-0778), itacitinib (also known as INCB039110, including the adipate salt), cerdulatinib (also known as PRT062070 and PRT2070), PF-06263276, delgotinib (also known as JTE-052), AS2553627, JAK-IN-35, ASN-002, AT9283, diosgenin, JAK inhibitor 1 (see US20170121327, compound example 283), JAK-IN-1, LFM-A13, NS-018 (including hydrochloride and maleate salts), RGB-286638, SB1317 (also known as TG02), curcumol, Go6976, JAK2 inhibitor G5-7, myricetin (also known as NSC 407290 and cannabiscetin), and pyridine 6 (also known as CMP6). For more description and chemical structures of exemplary JAK inhibitors, see, e.g., U.S. Pat. Nos. 9,198,911; 9,763,866; 9,737,469; 9,730,877; 9,895,301; 9,249,149; 9,518,027; 9,776,973; 9,549,367; and 9,931,343.

In some embodiments, a microsphere of the present disclosure comprises more than one therapeutic compound or pharmaceutically acceptable salt thereof. For example, a microsphere of the present disclosure can comprise multiple somatostatins, e.g., to target a particular somatostatin receptor profile in order to attain an altered pharmacodynamics effect.

In some embodiments, the dispersed phase comprises a polyol. Any of the polyols described herein may be used. In certain embodiments, the polyol comprises glycerol. In some embodiments, the polyol is present in the dispersed phase. In some embodiments, the polyol is solubilized in the dispersed phase.

In some embodiments, a microsphere of the present disclosure further comprises (or is made with a dispersed phase further comprising) a polyol. In some embodiments, the polyol comprises a (C₃₋₆) carbon chain containing alcohol having 2 to 6 hydroxyl groups and a mono- or di-saccharide, an esterified polyol having at least 3 polylactide-co-glycolide chains, glycols (e.g., propylene glycol with the number of OH reduced to 2), glucose, mannitol, or glycerol. In certain embodiments, the polyol is glycerol.

In some embodiments, the dispersed phase comprises the polyol at a concentration of between about 0.3 mg/mL and about 1.2 mg/mL. In some embodiments, the dispersed phase comprises the polyol at a concentration of between about 0.6 mg/mL and about 0.9 mg/mL. In some embodiments, the dispersed phase comprises the polyol at a concentration of about 0.9 mg/mL.

In some embodiments, a microsphere of the present disclosure is prepared according to a formulation described in Table A.

TABLE A
Microsphere formulations

| Formulation name | #73 | #121 | #131 | #137 | #139 | #154 | #175 | #173 |
|---|---|---|---|---|---|---|---|---|
| Actual loading (%) | 4.6 | 9.7 | 3.6 | 9.0 | 5.9 | 3.8 | 9.0 | 8.4 |
| PLGA 502h (%) | 0 | 100 | 0 | 75 | 50 | 0 | 75 | 65 |
| PLGA 503h (%) | 100 | 0 | 100 | 25 | 50 | 100 | 25 | 35 |
| PLGA concentration | 200 | 200 | 200 | 200 | 200 | 200 | 200 | 200 |
| (mg/ml)* | | | | | | | | |
| Octreotide acetate | 20 | 40 | 20 | 30 | 20 | 0 | 30 | 30 |
| (mg/ml)* | | | | | | | | |
| Octreotide benzoate | 0 | 0 | 0 | 0 | 0 | 20 | 0 | 0 |
| (mg/ml)* | | | | | | | | |
| Glycerol (mg/ml)* | 0 | 0 | 0.9 | 0 | 0 | 0 | 0.9 | 0 |
| Continuous phase | 100% | 100% | 100% | 100% | 100% | 100% | 100% | 100% |
| | DCM | DCM | DCM | DCM | DCM | DCM | DCM | DCM |
| | sat. | sat., | sat. | sat., | sat., | sat. | sat., | sat., |
| | 150 mM | 0.5% | 150 mM | 0.5% | 0.5% | 150 mM | 0.5% | 0.5% |
| | NaCl, | PVA, | NaCl, | PVA, | PVA, | NaCl, | PVA, | PVA, |
| | 0.5% | 200 mM | 0.5% | 200 mM | 200 mM | 0.5% | 200 mM | 200 mM |
| | PVA, | Gly | PVA, | Gly | Gly | PVA, | Gly | Gly |
| | 50 mM | pH 9.0 | 50 mM | pH 9.0 | pH 9.0 | 50 mM | pH 9.0 | pH 9.0 |
| | Gly, | | Gly | | | Gly, | | |
| | pH 8.5 | | pH 8.5 | | | pH 8.5 | | |
| Membrane pore size | 15 | 20 | 15 | 20 | 20 | 15 | 20 | 20 |
| (μm) | | | | | | | | |

*Final concentration in dispersed phase.

In some embodiments, the methods of the present disclosure further include adjusting the pH of the aqueous continuous phase. In some embodiments, the pH of the aqueous continuous phase is adjusted to the pI of the therapeutic compound or salt minus 0.5 or greater. In some embodiments, the pH of the aqueous continuous phase is adjusted to about 8 to about 9.5, e.g., to about 8, to about 8.5, to about 9, or to about 9.5. In some embodiments, the pH of the aqueous continuous phase is adjusted to about 7.5 to about 8.5, e.g., to about 7.5, to about 8, or to about 8.5. In some embodiments, the pH of the aqueous continuous phase is adjusted with a buffer solution. A variety of buffer solutions are known in the art and may be selected by one of skill in the art; exemplary buffers include, without limitation, glycine, glycylglycine, tricine, HEPES, MOPS, sulfonate, ammonia, potassium phosphate, CHES, borate, TAPS, Tris, bicine, TAPSO, TES, and Tris buffer solutions. In some embodiments, the buffer is glycylglycine, bicine, or tricine. In some embodiments, the buffer is not Tris buffer. In some embodiments, the pH of the aqueous continuous phase is adjusted to about 8.0 in glycylglycine, bicine, or tricine buffer. In some embodiments, the pH of the aqueous continuous phase is adjusted to about 8.0 in glycylglycine buffer.

In some embodiments, the continuous phase comprises ethanol, propanol, or methanol. In some embodiments, the continuous phase comprises dichloromethane, chloroform, or ethyl acetate.

In some embodiments, a continuous phase of the present disclosure comprises a surfactant. A variety of surfactants are known in the art and can be selected by one of skill in the art. In some embodiments, the surfactant is selected from polysorbate 20 or polysorbate 80 (e.g., of the TWEEN® series), poloxamer (e.g., of the PLURONIC® series; BASF), and polyvinyl alcohol (PVA). In some embodiments, the concentration of surfactant in the continuous phase is from 0.05% to 1% (w/w). In some embodiments, the concentration of surfactant in the continuous phase is at least about any of the following concentrations (in percentage, w/w): 0.05, 0.1, 0.2, 0.3, 0.4, 0.5, 0.6, 0.7, 0.8, or 0.9. In some embodiments, the concentration of surfactant in the continuous phase is less than about any of the following concentrations (in percentage, w/w): 1, 0.9, 0.8, 0.7, 0.6, 0.5, 0.4, 0.3, 0.2, or 0.1. That is, the concentration of surfactant in the continuous phase can be any concentration in a range having an upper limit of about 2, 1.5, 1, 0.9, 0.8, 0.7, 0.6, 0.5, 0.4, 0.3, 0.2, or 0.1% (w/w), and an independently selected lower limit of about 0.05, 0.1, 0.2, 0.3, 0.4, 0.5, 0.6, 0.7, 0.8, or 0.9% (w/w), wherein the upper limit is greater than the lower limit. In some embodiments, the concentration of surfactant in the continuous phase is about 0.5% (w/w).

The flowrates of the dispersed phase and continuous phase can vary depending on the application. In some embodiments, the flow rate of the continuous phase is at least about 0.01 liters/min, about 0.05 liters/min, about 0.1 liters/min, about 0.3 liters/min, about 0.5 liters/min, about 0.925 liters/min, about 1 liter/min, or about 5 liters/min. In some embodiments, the flow rate of the continuous phase can be about 0.01-5 liters/min, about 0.05-3 liters/min, about 0.1-2 liters/min, or about 0.5-1 liters/min. In some embodiments, the flow rate of the continuous phase is about 1.5 L/min to about 3.5 L/min or about 1.7 L/min to about 3.4 L/min. In some embodiments, the flow rate of the continuous phase is about 1.7 L/min, about 2.0 L/min, about 2.5 L/min, about 3.0 L/min, or about 3.4 L/min. In certain embodiments, the flow rate of the continuous phase is about 3.4 L/min.

In some embodiments, the flow rate of the dispersed phase can be at least about 0.1 mL/min, about 0.5 mL/min, about 0.75 mL/min, about 1 mL/min, about 1.25 mL/min, about 1.4 mL/min, about 2 mL/min, about 5 mL/min, or about 10 mL/min. In some embodiments, the flow rate of the dispersed phase can be about 0.1-10 mL/min, about 0.5-5 mL/min, about 1-2 mL/min, or about 1.4 mL/min. In some embodiments, the flow rate of the dispersed phase is about 8 mL/min to about 13 mL/min or about 9 mL/min to about 12 mL/min. In some embodiments, the flow rate of the dispersed phase is about 9 mL/min, about 10 mL/min, about 11 mL/min, or about 12 mL/min. In certain embodiments, the flow rate of the dispersed phase is about 10 mL/min. In certain embodiments, the flow rate of the dispersed phase is about 10 mL/min, and the flow rate of the continuous phase is about 3.4 L/min.

In some embodiments, a ratio of the flow of continuous phase to dispersed phase can be about 50:1 to about 5000:1, about 500:1 to about 3000:1, or about 750:1 to about 1000:1.

### Example 1

Applicants have created microspheres using the device disclosed herein and compared that with microspheres created using a commercial extrusion cell (LDC-1) from Micropore® that uses a paddle mixer to move the continuous phase. The comparative examples were created by the commercial Micropore® device with a stainless steel membrane with a 15 micron pore size and a 200 micron pore pitch. The inventive examples were prepared using a device disclosed herein with a stainless steel membrane with a 20 micron pore size and a 200 micron pore pitch. The continuous phase and the dispersed phase were the same in both the examples created by the device disclosed herein and the comparative examples created by the Micropore® device. The dispersed phase flow was 0.9 ml/min and the continuous phase flow was 935 ml/min.

The following table includes the formulations for the continuous phase and dispersed phase used to create the microspheres for the examples and comparative examples.

| | |
|---|---|
| Actual loading (%) | 4.6 |
| PLGA 503h (%) | 100 |
| PLGA concentration | 200 |
| (mg/ml)* | |
| Octreotide (mg/ml)* | 20 |
| as an acetate salt | |
| Continuous phase | 100% DCM sat. 150 mM NaCl, |
| | 0.5% PVA, 50 mM Glycine, pH |
| | 8.5 |

*Final concentration in dispersed phase.

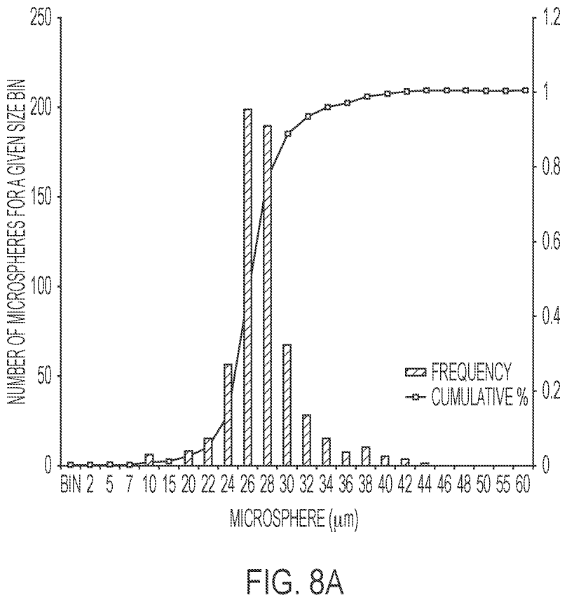

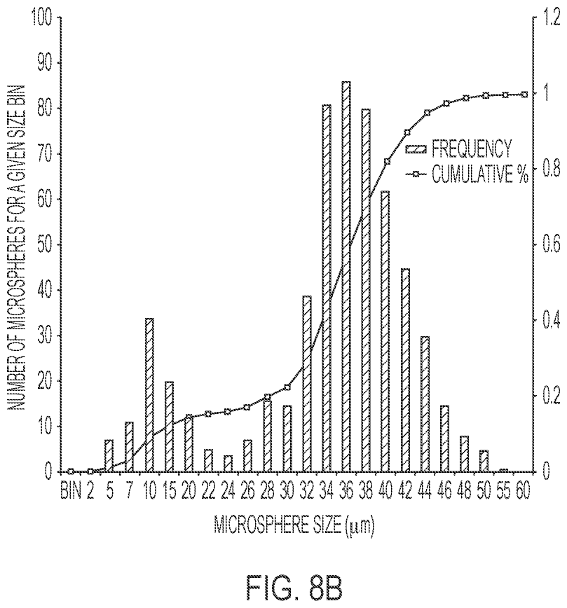

FIG. 8A is the microsphere size distribution created using the device disclosed herein. The microsphere size distribution can be calculated by a custom method in Cell Profiler® designed to find and measure spheres in light microscopy pictures. The key module for the method can be identifyPrimaryObjects and the measurements can be calibrated with a micrometric scale. FIG. 8B is the microsphere size distribution created using the Micropore® device. As shown by FIGS. 8A-B, the device disclosed herein improves size distribution of microspheres created compared to an extrusion cell with a paddle mixer.

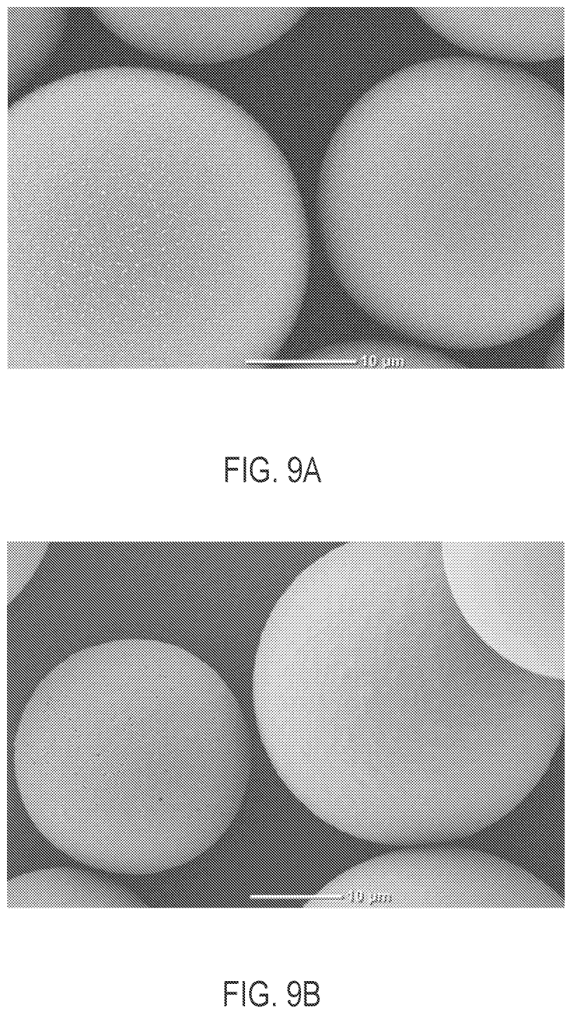

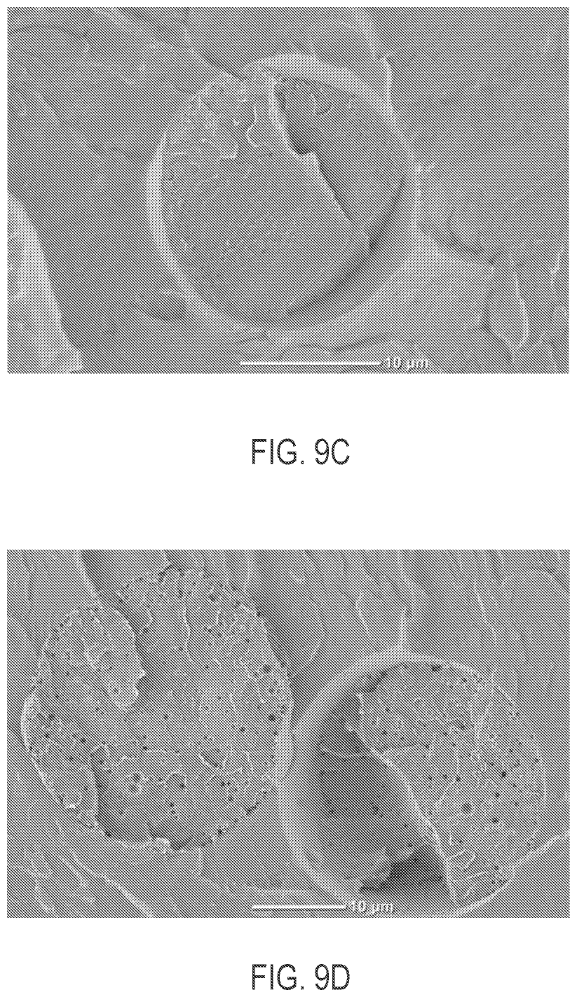

FIGS. 9A-B are SEM images taken of the microspheres created by the device disclosed herein. FIGS. 9C-D are SEM images taken of the microspheres created by the Micropore® device. As shown by FIGS. 9A-D, the appearance of the microspheres created by the device disclosed herein is drastically improved compared to the microspheres created by the Micropore® device. Specifically, the surface was improved (smoother, lack of pores) and the internal structure by cryofracture was improved.

### Example 2

This example describes a flow optimizing and scalable process to manufacture microspheres. The effects of batch size, continuous phase flow rate, and dispersed phase flow rate were also examined.

Briefly, microspheres were generated as follows. Octreotide was dissolved in ethanol, and PLGA was dissolved in methylene chloride. Both mixtures were then combined and extruded through calibrated pore-size membranes into a flowing continuous phase that strips the droplets from the surface of the membrane. The droplets were then hardened into microspheres with the continuous phase (without DCM), washed with water, and dried by lyophilization. The washing step may also include a “scalping” step whereby very small microspheres or “fines” may be further removed by selective filtration. This provides single emulsion microspheres with octreotide and PLGA.

Size distribution of microsphere populations was quantitated using automated image analysis. Cell Profiler (cellprofiler.org) software was used to find and measure microsphere diameter based on light microscopy images. The identifyPrimaryObjects module was used and calibrated with a micrometric scale. Starting with light microscopic images of microspheres, images were analyzed to automatically detect microsphere shape, fill in microsphere shapes, and measure microsphere dimensions.

In this example, the dispersed phase was prepared with 30 mg/mL octreotide acetate, 0.9 mg/mL glycerol with 150 mg/mL 502H, 50 mg/mL 503H PLGA dissolved in DCM. The continuous phase was prepared as 100 mM glycylglycine pH 8.0, 1% PVA, and saturated with DCM. A 10 μm membrane was used. The membrane emulsification device was cleaned and prepared using sodium hydroxide and citric acid. The system was equilibrated with continuous phase prior to use. FIG. 32 shows the directional flow for the continuous phase and dispersed phase.

50 mL of dispersed phase was pumped into the system (generating 10 grams of DP2018 microspheres) and the flow rates of the dispersed phase and continuous phase were varied. The microspheres were allowed to harden, washed with water and lyophilized. Dried microspheres were sized using a cell sizing program (Cell Profier) Lyophilized microspheres with a target diameter of ˜27 μm and with a size distribution comparable to microsphere manufactured with the Micropore Technologies Dispersion Cell were desired.

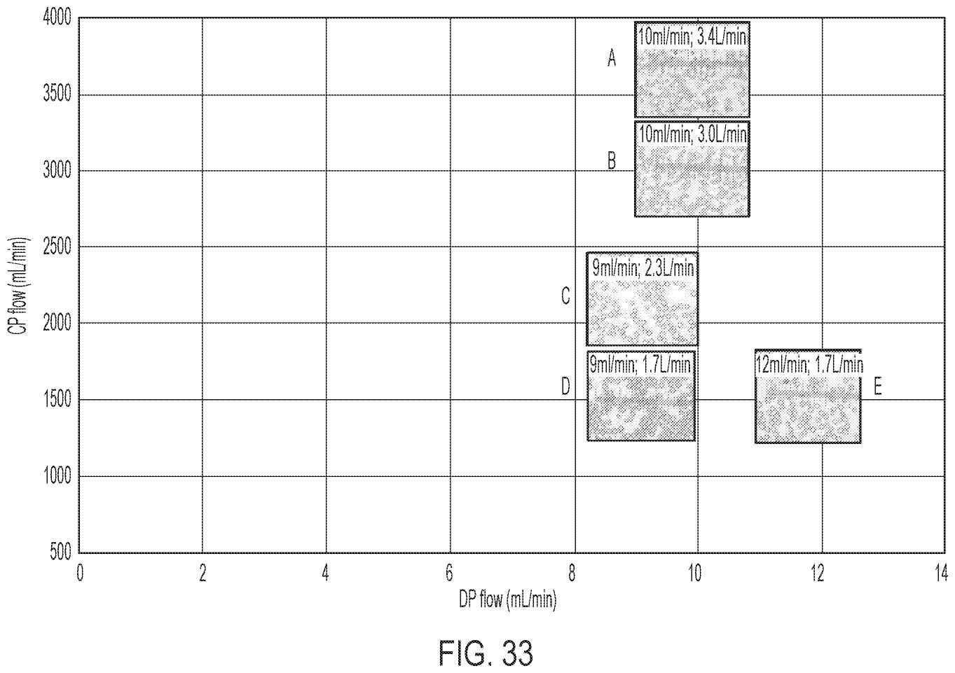

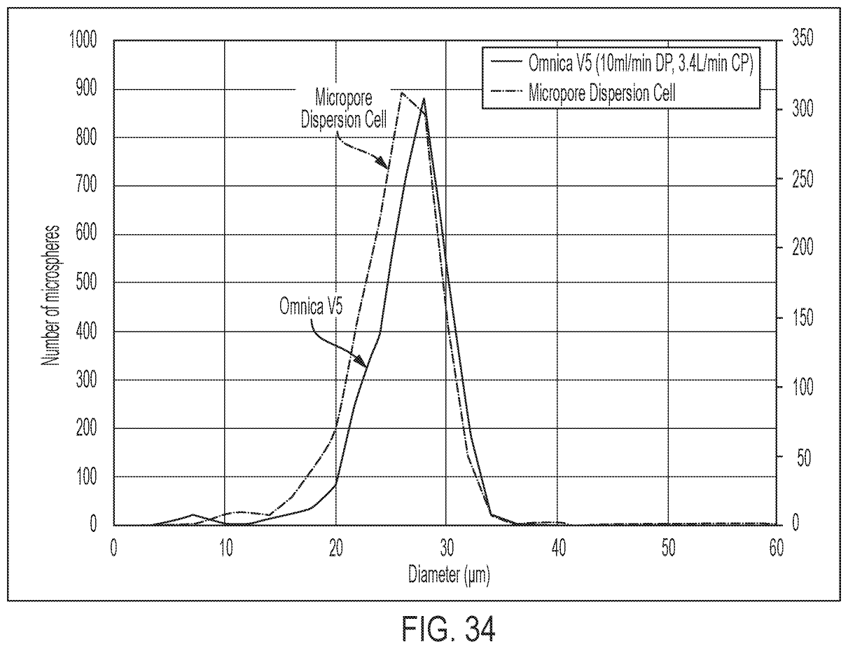

The correct combination of flow of P and DP must be applied so that correct size of microspheres are made without generating backflow into the dispersion plate side of the membrane. FIG. 33 shows the different microspheres that are generated at various continuous phase and dispersed phase flow rates. A flow rate of 10 ml/min for the dispersed phase and 3.4 L/min for the continuous phase generated the correct size of microspheres as shown in FIG. 34 which compares the size distribution of microspheres generated with the Micropore Dispersion Cell compared to the microspheres generated using the device disclosed herein.

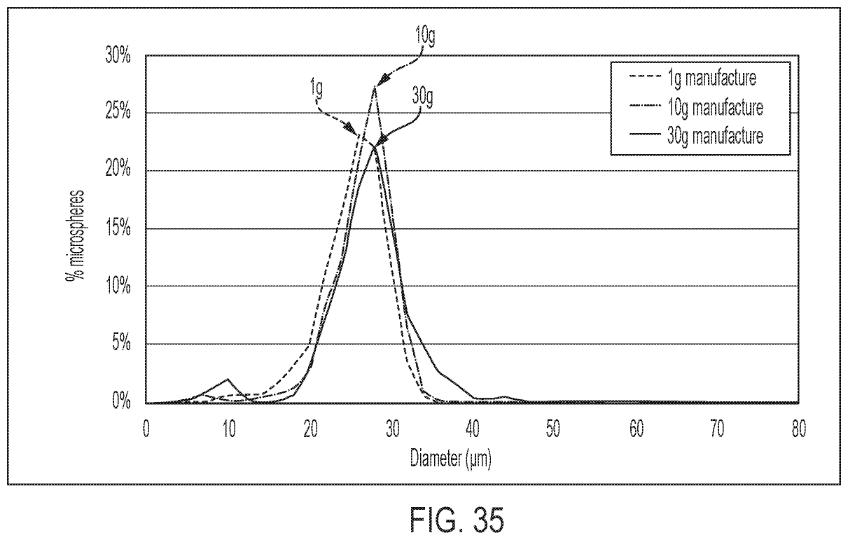

The extrusion can also be scaled up. Due to the constraints of the lab space, a 30 gram manufacture was the largest scale that could be run. As shown in FIG. 35, under the same flow rates for the dispersed phase and continuous phase for a 10 gram and 30 gram manufacture, microspheres with similar size distributions were manufactured. The 1 gram batch was made using the Micropore Dispersion Cell and added to the graph as a target distribution.

### Example 3

This example describes a reproducible process to manufacture microspheres.

For this example, the continuous phase and dispersed phase and device that were used were the same as those described with respect to example 2.

For a 10 gram batch, 50 mL of dispersed phase was used at a rate of 10 mL/min, with the flow rate of the continuous phase at 3.4 L/min. For a 30 gram batch, 150 mL of dispersed phase was used at a rate of 10 mL/min, with the flow rate of the continuous phase at 3.4 L/min. Microspheres were allowed to harden, washed with water, and dried. Dried microspheres were sized by quantitative image analysis of light field microscopic images (Cell Profiler).

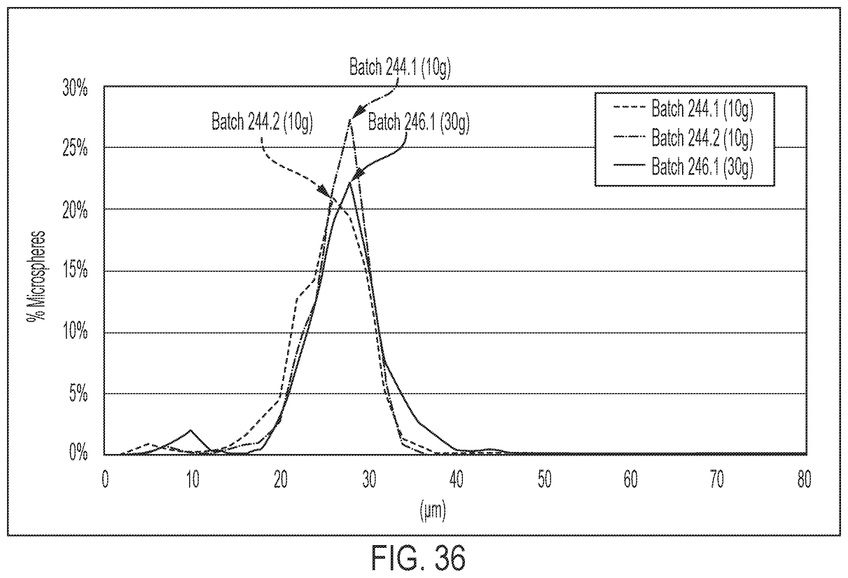

As shown in FIG. 36, two 10-gram and one 30-gram batches of microspheres had highly similar size distributions. 90-95% of the microspheres were 22-36 μm in diameter, with 60-70% at 26-34 μm in diameter. 20-30% were 28-32 μm.

### EMBODIMENTS

### Embodiment 1

A device, comprising:

a continuous phase plate comprising a continuous phase inlet;

a dispersed phase plate comprising a dispersed phase inlet;

an outlet; and

a chamber located between the continuous phase plate and the dispersed phase plate that is bisected by a membrane comprising a plurality of pores, wherein the chamber comprises:

at least one channel on a first side of the membrane formed from at least one groove in the continuous phase plate and the membrane, wherein the at least one channel is fluidly connected between the continuous phase inlet and the outlet; and

a cavity on a second side of the membrane formed in the dispersed phase plate that is fluidly connected between the dispersed phase inlet and the plurality of pores in the membrane.

### Embodiment 2

The device of embodiment 1, wherein the at least one channel extends in a direction transverse to a flow of a dispersed phase through the plurality of pores.

### Embodiment 3

The device of any of embodiments 1-2, wherein the continuous phase plate comprises the outlet.

### Embodiment 4

The device of any of embodiments 1-3, wherein the dispersed phase plate comprises a dispersed phase outlet.

### Embodiment 5

The device of any of embodiments 1-3, wherein the continuous phase plate comprises at least two grooves.

### Embodiment 6

The device of embodiment 5, wherein the chamber comprises at least two channels on the first side of the membrane formed from the at least two grooves in the continuous phase plate and the membrane.

### Embodiment 7

The device of any of embodiments 1-6, wherein the membrane is removably attached to the dispersed phase plate.

### Embodiment 8

The device of embodiment 7, wherein the continuous phase plate is removably attached to the dispersed phase plate.

### Embodiment 9

The device of any of embodiments 1-8, wherein the dispersed phase plate comprises a notch and the membrane is mounted in the notch.

### Embodiment 10

The device of any of embodiments 1-9, wherein the dispersed phase plate comprises stainless steel.

### Embodiment 11

The device of any of embodiments 1-10, wherein the continuous phase plate comprises stainless steel.

### Embodiment 12

The device of any of embodiments 1-11, wherein the membrane comprises alignment holes for mounting on the dispersed phase plate.

### Embodiment 13

The device of any of embodiments 1-12, wherein the membrane comprises stainless steel, tantalum, tungsten, molybdenum, manganese, tin, zinc, or an alloy thereof.

### Embodiment 14

The device of any of embodiments 1-13, wherein the membrane comprises porous glass or a ceramic.

### Embodiment 15

The device of any of embodiments 1-14, wherein one or more pores of the plurality of pores has a size between 10-50 microns.

### Embodiment 16

The device of embodiment 15, wherein one or more pores of the plurality of pores has a size between 10-20 microns.

### Embodiment 17

The device of any of embodiments 1-16, wherein the plurality of pores are uniformly sized.

### Embodiment 18

The device of any of embodiments 1-17, wherein the continuous phase plate, the dispersed phase plate, and the membrane are immobile.

### Embodiment 19

The device of any of embodiments 1-18, wherein the device does not have any moving device components.

### Embodiment 20

The device of any of embodiments 1-19, wherein a pressure drop between the continuous phase inlet and the outlet is smaller than the average pressure difference between the dispersed phase side of the membrane and the continuous phase side of the membrane.

### Embodiment 21

The device of any of embodiments 1-20, wherein a height or width of the at least one channel increases in a direction from the continuous phase inlet to the outlet.

### Embodiment 22

The device of any of embodiments 1-21, wherein a flow of the dispersed phase induces a pressure drop about equal to the pressure drop in the continuous phase at least one channel.

### Embodiment 23

A method of forming microspheres, comprising:

flowing a continuous phase through at least one channel of a chamber located between a continuous phase plate and a dispersed phase plate, the chamber bisected by a membrane comprising a plurality of pores, wherein the at least one channel is on a first side of the membrane and is formed from at least one groove in the continuous phase plate and the membrane;

forcing, on a second side of the membrane, a dispersed phase through the plurality of pores such that the dispersed phase enters into the continuous phase in a direction that is perpendicular to the continuous phase flow in the at least one channel,

wherein forcing the dispersed phase through the plurality of pores into the continuous phase forms a plurality of microspheres comprising the dispersed phase.

### Embodiment 24

The method of embodiment 23, wherein a median diameter of the plurality of microspheres is between 5-100 microns.

### Embodiment 25

The method of embodiment 24, wherein a median diameter of the plurality of microspheres is between 10-50 microns.

### Embodiment 26

The method of embodiment 25, wherein a median diameter of the plurality of microspheres is between 20-40 microns.

### Embodiment 27

The method of any of embodiments 23-26, wherein at least 70% of the plurality of microspheres have a diameter within 10 microns above or below the median diameter.

### Embodiment 28

The method of any of embodiments 23-27, wherein the coefficient of variation of a size distribution of the plurality of microspheres is less than 30%.

### Embodiment 29

The method of embodiment 28, wherein the coefficient of variation of a size distribution of the plurality of microspheres is less than 20%.

### Embodiment 30

The method of any of embodiments 23-29, wherein the coefficient of variation of a size distribution of the plurality of microspheres is between 10-20%.

### Embodiment 31

The method of embodiment 23-30, wherein the perpendicular flow of the continuous phase exerts a shear force at a shear rate at the membrane as the dispersed phase is forced through the plurality of pores.

### Embodiment 32

The method of embodiment 31, wherein the shear rate at the membrane is 1,000-25,000 s⁻¹.

### Embodiment 33

The method of any of embodiments 23-32, wherein the continuous phase comprises an aqueous solvent and the dispersed phase comprises an organic solvent.

### Embodiment 34

The method of embodiment 33, wherein the continuous phase further comprises a surfactant.

### Embodiment 35

The method of embodiment 34, wherein the dispersed phase further comprises a hydrophobic polymer.

### Embodiment 36

The method of any of embodiments 34-35, wherein the dispersed phase comprises a therapeutic compound or pharmaceutically acceptable salt thereof.

### Embodiment 37

The method of any of embodiments 34-36, wherein the dispersed phase comprises a polyol.

### Embodiment 38

The method of any of embodiments 23-37, wherein a pressure drop between the continuous phase flowing through the at least one channel on the first side of the membrane is smaller than a pressure of the dispersed phase on the second side of the membrane.

### Embodiment 39

The method of any of embodiments 23-38, wherein a height or width of the at least one channel increases in a flow direction of the continuous phase.

### Embodiment 40

The method of any of embodiments 23-39, wherein a flow of the dispersed phase induces a pressure drop about equal to the pressure drop in the continuous phase at least one channel.

### Embodiment 41

A device, comprising:

a continuous phase plate comprising a continuous phase inlet;

a dispersed phase plate comprising a plurality of dispersed phase inlets;

an outlet; and

a chamber located between the continuous phase plate and the dispersed phase plate that is bisected by a membrane comprising a plurality of pores, wherein the chamber comprises:

at least one channel on a first side of the membrane formed from at least one groove in the continuous phase plate and the membrane, wherein the at least one channel is fluidly connected between the continuous phase inlet and the outlet; and

a cavity on a second side of the membrane formed in the dispersed phase plate that is divided into a plurality of dispersed phase segments, wherein each of the dispersed phase segments are fluidly connected between a dispersed phase inlet and a plurality of pores in a portion of the membrane.

### Embodiment 42

The device of embodiment 41, wherein the dispersed phase segments are sequentially arranged along the length of the at least one channel.

### Embodiment 43

The device of embodiment 42, wherein a pressure of the dispersed phase in the sequential dispersed phase segments decreases along the length of the at least one channel.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that the membrane has at least about 1,000 pores, about 5,000 pores, or about 10,000 pores is meant to mean that the membrane has at least about can be less 1,000 pores, at least about 5,000 pores, or at least about 10,000 pores.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A device, comprising:
a continuous phase plate comprising a continuous phase inlet;
a dispersed phase plate comprising a dispersed phase inlet;
an outlet; and
a chamber located between the continuous phase plate and the dispersed phase plate that is bisected by a membrane comprising a plurality of pores, wherein the chamber comprises:
at least one channel on a first side of the membrane formed from at least one groove in the continuous phase plate and the membrane, wherein the at least one channel is fluidly connected between the continuous phase inlet and the outlet; and
a cavity on a second side of the membrane formed in the dispersed phase plate that is fluidly connected between the dispersed phase inlet and the plurality of pores in the membrane.

2. The device of claim 1, wherein the at least one channel extends in a direction transverse to a flow of a dispersed phase through the plurality of pores.

3. The device of any of claims 1-2, wherein the continuous phase plate comprises the outlet.

4. The device of any of claims 1-3, wherein the dispersed phase plate comprises a dispersed phase outlet.

5. The device of any of claims 1-3, wherein the continuous phase plate comprises at least two grooves.

6. The device of claim 5, wherein the chamber comprises at least two channels on the first side of the membrane formed from the at least two grooves in the continuous phase plate and the membrane.

7. The device of any of claims 1-6, wherein the membrane is removably attached to the dispersed phase plate.

8. The device of claim 7, wherein the continuous phase plate is removably attached to the dispersed phase plate.

9. The device of any of claims 1-8, wherein the dispersed phase plate comprises a notch and the membrane is mounted in the notch.

10. The device of any of claims 1-9, wherein the dispersed phase plate comprises stainless steel.

11. The device of any of claims 1-10, wherein the continuous phase plate comprises stainless steel.

12. The device of any of claims 1-11, wherein the membrane comprises alignment holes for mounting on the dispersed phase plate.

13. The device of any of claims 1-12, wherein the membrane comprises stainless steel, tantalum, tungsten, molybdenum, manganese, tin, zinc, or an alloy thereof.

14. The device of any of claims 1-13, wherein the membrane comprises porous glass or a ceramic.

15. The device of any of claims 1-14, wherein one or more pores of the plurality of pores has a size between 10-50 microns.

16. The device of claim 15, wherein one or more pores of the plurality of pores has a size between 10-20 microns.

17. The device of any of claims 1-16, wherein the plurality of pores are uniformly sized.

18. The device of any of claims 1-17, wherein the continuous phase plate, the dispersed phase plate, and the membrane are immobile.

19. The device of any of claims 1-18, wherein the device does not have any moving device components.

20. The device of any of claims 1-19, wherein a pressure drop between the continuous phase inlet and the outlet is smaller than the average pressure difference between the dispersed phase side of the membrane and the continuous phase side of the membrane.

21. The device of any of claims 1-20, wherein a height or width of the at least one channel increases in a direction from the continuous phase inlet to the outlet.

22. The device of any of claims 1-21, wherein a flow of the dispersed phase induces a pressure drop about equal to the pressure drop in the continuous phase at least one channel.

23. A method of forming microspheres, comprising:
flowing a continuous phase through at least one channel of a chamber located between a continuous phase plate and a dispersed phase plate, the chamber bisected by a membrane comprising a plurality of pores, wherein the at least one channel is on a first side of the membrane and is formed from at least one groove in the continuous phase plate and the membrane;
forcing, on a second side of the membrane, a dispersed phase through the plurality of pores such that the dispersed phase enters into the continuous phase in a direction that is perpendicular to the continuous phase flow in the at least one channel,
wherein forcing the dispersed phase through the plurality of pores into the continuous phase forms a plurality of microspheres comprising the dispersed phase.

24. The method of claim 23, wherein a median diameter of the plurality of microspheres is between 5-100 microns.

25. The method of claim 24, wherein a median diameter of the plurality of microspheres is between 10-50 microns.

26. The method of claim 25, wherein a median diameter of the plurality of microspheres is between 20-40 microns.

27. The method of any of claims 23-26, wherein at least 70% of the plurality of microspheres have a diameter within 10 microns above or below the median diameter.

28. The method of any of claims 23-27, wherein the coefficient of variation of a size distribution of the plurality of microspheres is less than 30%.

29. The method of claim 28, wherein the coefficient of variation of a size distribution of the plurality of microspheres is less than 20%.

30. The method of any of claims 23-29, wherein the coefficient of variation of a size distribution of the plurality of microspheres is between 10-20%.

31. The method of claim 23-30, wherein the perpendicular flow of the continuous phase exerts a shear force at a shear rate at the membrane as the dispersed phase is forced through the plurality of pores.

32. The method of claim 31, wherein the shear rate at the membrane is 1,000-25,000 s⁻¹.

33. The method of any of claims 23-32, wherein the continuous phase comprises an aqueous solvent and the dispersed phase comprises an organic solvent.

34. The method of claim 33, wherein the continuous phase further comprises a surfactant.

35. The method of claim 34, wherein the dispersed phase further comprises a hydrophobic polymer.

36. The method of any of claims 34-35, wherein the dispersed phase comprises a therapeutic compound or pharmaceutically acceptable salt thereof

37. The method of any of claims 34-36, wherein the dispersed phase comprises a polyol.

38. The method of any of claims 23-37, wherein a pressure drop between the continuous phase flowing through the at least one channel on the first side of the membrane is smaller than a pressure of the dispersed phase on the second side of the membrane.

39. The method of any of claims 23-38, wherein a height or width of the at least one channel increases in a flow direction of the continuous phase.

40. The method of any of claims 23-39, wherein a flow of the dispersed phase induces a pressure drop about equal to the pressure drop in the continuous phase at least one channel.

41. A device, comprising:
a continuous phase plate comprising a continuous phase inlet;
a dispersed phase plate comprising a plurality of dispersed phase inlets;
an outlet; and
a chamber located between the continuous phase plate and the dispersed phase plate that is bisected by a membrane comprising a plurality of pores, wherein the chamber comprises:
at least one channel on a first side of the membrane formed from at least one groove in the continuous phase plate and the membrane, wherein the at least one channel is fluidly connected between the continuous phase inlet and the outlet; and
a cavity on a second side of the membrane formed in the dispersed phase plate that is divided into a plurality of dispersed phase segments, wherein each of the dispersed phase segments are fluidly connected between a dispersed phase inlet and a plurality of pores in a portion of the membrane.

42. The device of claim 41, wherein the dispersed phase segments are sequentially arranged along the length of the at least one channel.

43. The device of claim 42, wherein a pressure of the dispersed phase in the sequential dispersed phase segments decreases along the length of the at least one channel.
